(19) United States
(12) Patent Application Publication
(10) Pub. No.: US 2004/0203083 A1
(43) Pub. Date: Oct. 14, 2004

(54) Use of thrombus precursor protein and monocyte chemoattractant protein as diagnostic and prognostic indicators in vascular diseases

(75) Inventors: Kenneth F. Buechler, Rancho Santa Fe, CA (US); Alan Maisel, Solana Beach, CA (US)

Correspondence Address: FOLEY & LARDNER, P.O. BOX 80278, SAN DIEGO, CA, 92138-0278, US

(73) Assignee: Biosite, Inc.

(21) Appl. No.: 10/728,067

(22) Filed: Dec. 3, 2003

Related U.S. Application Data
(63) Continuation-in-part of application No. 10/603,891, filed on Jun. 24, 2003.
Application No. 10/603,891 is a continuation-in-part of application No. 10/330,696, filed on Dec. 27, 2002.
(63) Continuation-in-part of application No. 10/139,086, filed on May 4, 2002.
(63) Continuation-in-part of application No. 09/835,298, filed on Apr. 13, 2001.
(63) Continuation-in-part of application No. 10/225,082, filed on Aug. 20, 2002.
(60) Provisional application No. 60/436,301, filed on Dec. 24, 2002.
(60) Provisional application No. 60/288,871, filed on May 4, 2001.
(60) Provisional application No. 60/315,642, filed on Aug. 28, 2001.
(60) Provisional application No. 60/313,775, filed on Aug. 20, 2001.
(60) Provisional application No. 60/334,964, filed on Nov. 30, 2001.
(60) Provisional application No. 60/346,485, filed on Jan. 2, 2002.

(51) Int. Cl. G01N 33/53; G01N 33/537; G01N 33/543

(52) U.S. Cl. 435/7.92

(57) ABSTRACT

The present invention relates to methods for the diagnosis and evaluation of acute coronary syndromes. In particular, patient test samples are analyzed for the presence and amount of members of a panel of markers comprising one or more specific markers for myocardial injury and one or more non-specific markers for myocardial injury. A variety of markers are disclosed for assembling a panel of markers for such diagnosis and evaluation. In various aspects, the invention provides methods for the early detection and differentiation of stable angina, unstable angina, and myocardial infarction. Invention methods provide rapid, sensitive and specific assays that can greatly increase the number of patients that can receive beneficial treatment and therapy, reduce the costs associated with incorrect diagnosis, and provide important information about the prognosis of the patient.

This application is a continuation in part of U.S. patent application Ser. No. 10/603,891, filed Jun. 24, 2003, which is a continuation in part of U.S. patent application Ser. No. 10/330,696 filed Dec. 27, 2002, and which claims priority to U.S. Provisional Patent Application No. 60/436,301 filed Dec. 24, 2002; a continuation in part of U.S. patent application Ser. No. 10/139,086 filed May 4, 2002, which claims priority to U.S. Provisional Patent Application No. 60/315,642 filed Aug. 28, 2001, and to U.S. Provisional Patent Application No. 60/288,871 filed May 4, 2001; a continuation in part of U.S. patent application Ser. No. 09/835,298 filed Apr. 13, 2001; and a continuation in part of U.S. patent application Ser. No. 10/225,082 filed Aug. 20, 2002, which claims priority to U.S. Provisional Patent Application No. 60/346,485 filed Jan. 2, 2002, to U.S. Provisional Patent Application No. 60/334,964 filed Nov. 30, 2001, and to U.S. Provisional Patent Application No. 60/313,775 filed Aug. 20, 2001; from each of which priority is claimed, and each of which is hereby incorporated by reference in its entirety, including all tables, figures, and claims.

### FIELD OF THE INVENTION

The present invention relates to the identification and use of diagnostic and prognostic markers for vascular diseases, particularly acute coronary syndromes (ACS) and stroke. In various aspects, the invention relates to methods for the early detection and differentiation vascular diseases and the identification of individuals at risk for adverse events upon presentation with symptoms of vascular disease.

### BACKGROUND OF THE INVENTION

The following discussion of the background of the invention is merely provided to aid the reader in understanding the invention and is not admitted to describe or constitute prior art to the present invention.

Vascular diseases are the leading cause of morbidity and mortality in the United States and most western countries. According to 1994 statistics, vascular disease accounted for twice as many deaths in the U.S. compared to cancers, and ten times as many deaths as accidents. Atherosclerosis is the most common vascular disease, but the term “vascular diseases” as used herein includes stroke (both ischemic stroke, intracerebral hemorrhage, and subarachnoid hemorrhage), transient ischemic attack, systolic dysfunction, diastolic dysfunction, aneurysm (including aortic dissections), myocardial ischemia (also called “coronary artery disease”), angina pectoris, myocardial infarction, congestive heart failure, cardiomyopathy (including dilated congestive cardiomyopathy, hypertrophic cardiomyopathy, and restrictive cardiomyopathy), cor pulmonale, arrhythmias, valvular heart disease, endocarditis, and peripheral vascular disease (including peripheral arterial occlusion and venous thrombosis).

The term “acute coronary syndromes” (“ACS”) has been applied to a group of vascular diseases that result from ischemic insult to the heart. ACS is a manifestation of vascular injury to the heart, also referred to as myocardial injury or myocardial damage, that is commonly secondary to atherosclerosis or hypertension, and is the leading cause of death in the United States. ACS is commonly caused by occlusion associated with coronary artery disease cause by atherosclerotic plaque formation and progression to either further occlusion or fissure. ACS can be manifested as stable angina, unstable angina, or myocardial infarction.

Patients with ACS form a heterogeneous group, with differences in pathophysiology, clinical presentation, and risk for adverse events. Such patients present to the physician with conditions that span a continuum that includes unstable angina, non-ST-elevation non-Q wave myocardial infarction (“NST”-“MI”), ST-elevation non-Q wave MI, and transmural (Q-wave) MI. ACS is believed to result largely from thrombus deposition and growth within one or more coronary arteries, resulting in a partial or complete occlusion of the artery, and frequently involves rupture of the plaque, resulting in an ischemic injury. ACS may also be precipitated by a coronary vasospasm or increased myocardial demand. For review, see, e.g., Davies, Clin. Cardiol. 20 (Supp. I): I2-I7 (1997).

The seriousness of ACS is underlined by the morbidity and mortality that follow the ischemic insult. For example, workers have estimated that within four to six weeks of presentation with ACS, the risk of death or a subsequent myocardial infarction (MI) is 8-14%, and the rate of death, MI, or refractory ischemia is 15-25% (Theroux and Fuster, Circulation 97: 1195-1206, 1998). Given that the total number of deaths in the U.S. from acute MI is about 600,000, the search within the art for information that relates to the diagnosis, prognosis, and management of ACS has understandably been extensive. Several potential markers that may provide such information in certain patient populations have been identified, including circulating cardiac troponin levels (see, e.g., Antman et al., N. Eng. J. Med. 335: 1342-9, 1996; see also U.S. Pat. Nos. 6,147,688, 6,156,521, 5,947,124, and 5,795,725, each of which is hereby incorporated by reference in its entirety), ST-segment depression (see, e.g., Savonitto et al., JAMA 281: 707-13, 1999), circulating creatine kinase levels (see, e.g., Alexander et al., Circulation (Suppl.) 1629, 1998), and circulating c-reactive protein levels (see, e.g., Morrow et al., J. Am. Coll. Cardiol. 31: 1460-5, 1998).

Stable angina is characterized by constricting chest pain that occurs upon exertion or stress, and is relieved by rest or sublingual nitroglycerin. Unstable angina is characterized by constricting chest pain at rest that is relieved by sublingual nitroglycerin. Anginal chest pain is usually relieved by sublingual nitroglycerin, and the pain usually subsides within 30 minutes. Myocardial infarction is characterized by constricting chest pain lasting longer than 30 minutes that can be accompanied by diagnostic electrocardiography (ECG) Q waves. Unstable angina is thought to represent the clinical state between stable angina and myocardial infarction, and is commonly associated with atherosclerotic plaque rupture and thrombus formation. In this regard, atherosclerotic plaque rupture is the most common cause of myocardial infarction.

Inflammation occurs during stable angina, and markers of plaque rupture, platelet activation, and early thrombosis can be used to identify and monitor the progressing severity of unstable angina. The myocardial damage caused during an anginal attack is, by definition, reversible, while damage caused during a myocardial infarction is irreversible. According to this model, a specific marker of myocardial injury can be used to identify myocardial infarction. The progression of coronary artery disease from mild unstable angina to severe unstable angina and myocardial infarction is related to plaque instability and the degree of arterial occlusion. This progression can occur slowly, as stable plaques enlarge and become more occlusive, or it can occur rapidly, as unstable plaques rupture, causing platelet activation and occlusive thrombus formation. Because myocardial infarction most frequently shares the same pathophysiology as unstable angina, it is possible that the only distinction between these two events is the reversibility of myocardial damage. However, since the only distinction between severe unstable angina and mild myocardial infarction is based on clinical judgement, markers of myocardial damage may also appear in the peripheral circulation of patients diagnosed as having unstable angina.

Current diagnostic methods for ACS commonly include clinical symptoms, electrocardiography (ECG), and the measurement of cardiac markers in the peripheral circulation. Angiography is also used in cases of severe chest pain usually associated with unstable angina and acute myocardial infarction (AMI). Patients with ACS frequently have constricting chest pain that often radiates to the neck, jaw, shoulders, or down the inside of the left or both arms and can have accompanying symptoms of dyspnea, diaphoresis, palpitations, light-headedness, and nausea. Myocardial ischemia can produce diagnostic ECG changes including Q waves and ST segment changes. Elevations of the plasma concentration of cardiac enzymes may reflect the degree of cardiac tissue necrosis associated with severe unstable angina and myocardial infarction.

### SUMMARY OF THE INVENTION

The present invention relates to the identification and use of diagnostic and/or prognostic markers for one or more vascular diseases. The methods and compositions described herein can meet the need in the art for a rapid, sensitive and specific diagnostic assay to be used in the diagnosis, differentiation and prognosis of various vascular diseases. Moreover, the methods and compositions of the present invention can also be used to facilitate the treatment of patients and the development of additional diagnostic indicators.

As described herein, thrombus precursor protein (“TpPTM”) and monocyte chemoattractant protein-1 (MCP-1) each represents an independent marker for use in risk stratification and diagnosis of patients suffering from vascular diseases. In the case of ACS for example, TpP™ and/or MCP-1 may permit a determination of risk that a subject may suffer from a future clinical outcome such as death, nonfatal myocardial infarction, recurrent ischemia requiring urgent revascularization, and/or recurrent ischemia requiring rehospitalization.

The time horizon over which such risk stratification may be applied (that is, the period for which prognostic risk may be predicted) may be from 1 day to 5 years, more preferably from 1 week to 2 years, and most preferably from 1 month to 1 year. While described hereinafter with regard to ACS patients, TpP™ and MCP-1 may also be used in various aspects according to the methods described herein to provide diagnostic and prognostic information in a variety of vascular diseases in which coagulation and hemostasis and/or inflammation are implicated.

Preferred diseases to which the various aspects described herein may be applied include one or more diseases selected from the group consisting of sepsis, acute coronary syndrome, atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, and peripheral vascular disease.

In a first aspect, the invention features methods of predicting a risk of one or more clinical outcomes for a subject suffering from a vascular disease by analyzing a test sample obtained from the subject for the presence or amount of TpP™ and/or MCP-1, and using the presence or amount of TpP™ and/or MCP-1 measured in the sample to associate a risk of one or more clinical outcomes to the subject.

As described hereinafter, TpP™ and/or MCP-1 may be associated with a given risk of one or more clinical outcomes without considering any other markers. Thus, in certain embodiments, such an association may be made simply by providing one or more predetermined threshold concentrations, below which a subject has a first risk level, and above which a subject has a second risk level. A subject may be assigned a a relative prognostic risk based upon a population of vascular disease patients for whom TpP™ and/or MCP-1 concentrations have been measured, and subsequent clinical outcomes followed over a period of days, months, or years. The population TpP™ and/or MCP-1 (as relevant) concentrations may be divided into tertiles, quartiles, quintiles, etc., and an associated risk level determined for each subpopulation by methods known in the art. Patients may then be assigned to one of these prognostic risk subpopulations according to a measured TpPTM and/or MCP-1 concentration.

In other embodiments, TpP™ and/or MCP-1 are used as part of panels as described hereinafter to associate a risk of one or more clinical outcomes to the subject. Such panels may comprise 2, 3, 4, 5, 6, 7, 8, 9, 10, 15, 20, or more or individual markers, at least one of which is TpP™ or MCP-1. Preferred panels comprise a plurality of markers independently selected from the group consisting of TpP™, MCP-1, and one or more additional markers independently selected from the group consisting of specific markers of cardiac injury, specific markers of neural tissue injury, markers related to blood pressure regulation, markers related to inflammation, markers related to coagulation and hemostasis, and markers related to apoptosis. Exemplary markers in each of these groups are described hereinafter. One or more markers considered with TpP™ and/or MCP-1 may lack diagnostic or prognostic value when considered alone, but when used as part of a panel, such markers may be of great value in determining a particular diagnosis and/or prognosis.

Preferred marker(s) related to blood pressure regulation for use in the methods described herein comprise, for example, one or more marker(s) selected from the group consisting of atrial natriuretic peptide (“ANP”), pro-ANP, B-type natriuretic peptide (“BNP”), NT-pro BNP, pro-BNP C-type natriuretic peptide, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto.

Preferred marker(s) markers related to inflammation for use in the methods described herein comprise, for example, one or more marker(s) selected from the group consisting of acute phase reactants, cell adhesion molecules such as vascular cell adhesion molecule (“VCAM”), intercellular adhesion molecule-1 (“ICAM-1”), intercellular adhesion molecule-2 (“ICAM-2”), and intercellular adhesion molecule-3 (“ICAM-3”), C-reactive protein, interleukins such as IL-1β, IL-6, and IL-8, interleukin-1 receptor agonist, caspase-3, lipocalin-type prostaglandin D synthase, mast cell tryptase, eosinophil cationic protein, KL-6, haptoglobin, tumor necrosis factor α, tumor necrosis factor β, Fas ligand, soluble Fas (Apo-1), TRAIL, TWEAK, fibronectin, macrophage migration inhibitory factor (MIF), and vascular endothelial growth factor (“VEGF”), or markers related thereto. The term “acute phase reactants” as used herein refers to proteins whose concentrations are elevated in response to stressful or inflammatory states that occur during various insults that include infection, injury, surgery, trauma, tissue necrosis, and the like. Acute phase reactant expression and serum concentration elevations are not specific for the type of insult, but rather as a part of the homeostatic response to the insult.

In addition to those acute phase reactants listed above as “markers related to inflammation,” one or more markers related to inflammation may also be selected from the group of acute phase reactants consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, e-selectin, glutathione-S-transferase, hypoxia-inducible factor-1α, inducible nitric oxide synthase (“I—NOS”), intracellular adhesion molecule, lactate dehydrogenase, monocyte chemoattractant peptide-1 (“MCP-1”), n-acetyl aspartate, prostaglandin E2, receptor activator of nuclear factor (“RANK”) ligand, TNF receptor superfamily member 1A, lipopolysaccharide binding protein (“LBP”), and cystatin C, or markers related thereto.

Preferred marker(s) related to coagulation and hemostasis for use in the methods described herein comprise, for example, one or more marker(s) selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, and von Willebrand factor, tissue factor, or markers related thereto.

Preferred marker(s) related to apoptosis for use in the methods described herein comprise, for example, one or more marker(s) selected from the group consisting of spectrin, cathepsin D, caspase 3, s-acetyl glutathione, and ubiquitin fusion degradation protein 1 homolog.

Suitable additional markers for inclusion in such panels are described in detail hereinafter. Particularly preferred markers for use in such panels in addition to TpP™ include BNP, cardiac troponin I (free and/or complexed), cardiac troponin T (free and/or complexed), CRP, creatine kinase-MB, MMP-9, caspase-3, myoglobin, myeloperoxidase, sCD40L, or markers related thereto. One or more markers may be replaced, added, or subtracted from this list of particularly preferred markers while still providing clinically useful results.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker such a value; alternatively, if only a subset of markers are outside of a normal range, this subset may be indicative of a particular diagnosis/prognosis. For example, certain markers in a panel may be used to diagnose (or to rule out) a myocardial infarction, while other members of the panel may diagnose (or rule out) congestive heart failure, while still other members of the panel may diagnose (or rule out) aortic dissection. Markers may also be commonly used for multiple purposes by, for example, applying a different threshold or a different weighting factor to the marker for the different purpose(s). For example, a marker at one concentration or weighting may be used, alone or as part of a larger panel, to to diagnose (or to rule out) a myocardial infarction, and the same marker at a different concentration or weighting may be used, alone or as part of a larger panel, to diagnose (or rule out) congestive heart failure, etc.

In certain embodiments, one or more diagnostic or prognostic indicators are correlated to a condition or disease by merely the presence or absence of the indicator(s). For example, an assay can be designed so that a positive signal for a marker only occurs above a particular threshold concentration of interest, and below which concentration the assay provides no signal above background. In other embodiments, threshold concentration(s) of diagnostic or prognostic indicator(s) can be established, and the level of the indicator(s) in a patient sample can simply be compared to the threshold level(s).

The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982). Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

In certain embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile as a unitary whole. A particular “fingerprint” pattern of changes in such a panel of markers may, in effect, act as a specific diagnostic or prognostic indicator. As discussed herein, that pattern of changes may be obtained from a single sample, or from temporal changes in one or more members of the panel (or a panel response value).

As described herinafter, a panel response value is preferably determined by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) of a diagnosis or prognosis. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of atest's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

The skilled artisan will understand that associating a diagnostic or prognostic indicator, with a diagnosis or with a prognostic risk of a future clinical outcome is a statistical analysis. For example, a marker level of greater than X may signal that a patient is more likely to suffer from an adverse outcome than patients with a level less than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In yet other embodiments, multiple determinations of one or more diagnostic or prognostic markers can be made, and a temporal change in the marker can be used to determine a diagnosis or prognosis. For example, a marker concentration in a subject sample may be determined at an initial time, and again at a second time from a second subject sample. In such embodiments, an increase in the marker from the initial time to the second time may be indicative of a particular diagnosis, or a particular prognosis. Likewise, a decrease in the marker from the initial time to the second time may be indicative of a particular diagnosis, or a particular prognosis. This “temporal change” parameter can be included as a marker in the marker panels of the present invention.

In a related embodiment, multiple determinations of one or more diagnostic or prognostic markers can be made, and a temporal change in the marker can be used to monitor the efficacy of appropriate therapies. In such an embodiment, one might expect to see a decrease or an increase in the marker(s) over time during the course of effective therapy.

The skilled artisan will understand that, while in certain embodiments comparative measurements are made of the same diagnostic marker at multiple time points, one could also measure a given marker at one time point, and a second marker at a second time point, and a comparison of these markers may provide diagnostic information. Similarly, the skilled artisan will understand that serial measurements and changes in markers or the combined result over time may also be of diagnostic and/or prognostic value.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In another aspect of the present invention, methods of diagnosing a vascular disease are described. Such methods comprise analyzing a test sample obtained from the subject for the presence or amount of TpP™ and/or MCP-1 and one or more additional markers, and using the presence or amount of TpP™ and/or MCP-1 and the additional marker(s) to determine the presence or absence of the vascular disease in the subject. In this aspect then, TpP™ and/or MCP-1 is used as part of a diagnostic panel. As above, such panels may comprise 2, 3, 4, 5, 6, 7, 8, 9, 10, 15, 20, or more or individual markers, at least one of which is TpP™ or MCP-1. Preferred panels comprise a TpP™ and/or MCP-1 and one or more additional markers independently selected from the group consisting of specific markers of cardiac injury, specific markers of neural tissue injury, markers related to blood pressure regulation, markers related to inflammation, markers related to coagulation and hemostasis, and markers related to apoptosis.

In a related aspect of the present invention, methods of diagnosing atherosclerosis are described. Such methods comprise analyzing a test sample obtained from the subject for the presence or amount of MCP-1 (and optionally one or more additional markers), and using the presence or amount of MCP-1 (and the additional marker(s) if measured) to determine the presence or absence of atherosclerosis in the subject. When MCP-1 is used as part of a diagnostic panel in this aspect, such panels may comprise 2, 3, 4, 5, 6, 7, 8, 9, 10, 15, 20, or more or individual markers, at least one of which is MCP-1. Preferred panels comprise MCP-1 and one or more additional markers independently selected from the group consisting of specific markers of cardiac injury, specific markers of neural tissue injury, markers related to blood pressure regulation, markers related to inflammation, markers related to coagulation and hemostasis, and markers related to apoptosis.

In yet another aspect, the invention relates to methods for determining a treatment regimen for use in a subject. The methods preferably comprise determining a diagnosis or prognosis as described herein, and selecting one or more treatment regimens that improve the patient's prognosis by reducing the increased disposition for an adverse outcome associated with the diagnosis. Such methods may also be used to screen pharmacological compounds for agents capable of improving the patient's prognosis as above.

In a further aspect, the invention relates to kits and devices for determining the diagnosis or prognosis of a patient. Kits preferably comprise devices and reagents for performing the assays described herein, and instructions for performing the assays. Optionally, the kits may contain one or more means for converting marker level(s) to a diagnosis or prognosis. Such kits preferably contain sufficient reagents to perform one or more such determinations, and/or Food and Drug Administration (FDA)-approved labeling.

### BRIEF DESCRIPTION OF THE FIGURES

FIG. 1 shows the relationship of TpP™ concentration to clinical outcome through 12 months following enrollment of ACS subjects in the OPUS-TIMI 16 study.

FIG. 2 shows the relationship of MCP-1 concentration to atherosclerosis in subjects not exhibiting clinically apparent atherosclerosis as measured by determining CAC.

### DETAILED DESCRIPTION OF THE INVENTION

In accordance with the present invention, there are provided methods and compositions for the identification and use of markers that are associated with the diagnosis, prognosis, or differentiation of vascular disease in a patient. Such markers can be used in diagnosing and treating a patient and/or to monitor the course of a treatment regimen; and for screening compounds and pharmaceutical compositions that might provide a benefit in treating or preventing such conditions. While described herein with regard to acute coronary syndromes, the methods and compositions described herein may be applied to any vascular diseases in which coagulation and hemostasis are implicated.

The Acute Coronary Syndrome

Myocardial ischemia is caused by an imbalance of myocardial oxygen supply and demand. Specifically, demand exceeds supply due to inadequate blood supply. The heart accounts for a small percentage of total body weight, but is responsible for 7% of body oxygen consumption. Cardiac tissue metabolism is highly aerobic and has very little reserve to compensate for inadequate blood supply. When the blood supply is reduced to levels that are inadequate for myocardial demand, the tissue rapidly becomes hypoxic and toxic cellular metabolites can not be removed. Myocardial cells rapidly use oxygen supplies remaining in the local microvasculature, and the length of time that aerobic metabolism continues is indirectly proportional to the degree of arterial occlusion. Once the oxygen supply has been exhausted, oxidative phosphorylation can not continue because oxygen is no longer available as an electron acceptor, pyruvate can not be converted to acetyl coenzyme A and enter the citric acid cycle. Myocardial metabolism switches to anaerobic metabolism using glycogen and glucose stores, and pyruvate is fermented to lactate. Lactate accumulation is the primary cause of chest pain in individuals with ACS. As ischemia continues, cardiac tissue becomes more acidic as lactate and other acidic intermediates accumulate, ATP levels decrease, and available energy sources are depleted. Cardiac tissue can recover if it is reperfused 15-20 minutes after an ischemic event. After the cellular glycogen stores have been depleted, the cell gradually displays features of necrosis, including mitochondrial swelling and loss of cell membrane integrity. Upon reperfusion, these damaged cells die, possibly as a result of the cell's inability to maintain ionic equilibrium. A loss of membrane integrity causes the cell's cytosolic contents to be released into the circulation.

Stable angina, unstable angina, and myocardial infarction all share one common feature: constricting chest pain associated with myocardial ischemia. Angina is classified as stable or unstable through a physician's interpretation of clinical symptoms, with or without diagnostic ECG changes. The classification of angina as “stable” or “unstable” does not refer to the stability of the plaque itself, but rather, the degree of exertion that is required to elicit chest pain. Most notably, the classification of chest pain as stable or unstable angina (or even mild myocardial infarction) in cases other than definitive myocardial infarction is completely subjective. The diagnosis, and in this case the distinction, is made not by angiography, which may quantify the degree of arterial occlusion, but rather by a physician's interpretation of clinical symptoms.

Stable angina is characterized by constricting chest pain that occurs upon exertion or stress, and is relieved by rest or sublingual nitroglycerin. Coronary angiography of patients with stable angina usually reveals 50-70% obstruction of at least one coronary artery. Stable angina is usually diagnosed by the evaluation of clinical symptoms and ECG changes. Patients with stable angina may have transient ST segment abnormalities, but the sensitivity and specificity of these changes associated with stable angina are low.

Unstable angina is characterized by constricting chest pain at rest that is relieved by sublingual nitroglycerin. Anginal chest pain is usually relieved by sublingual nitroglycerin, and the pain usually subsides within 30 minutes. There are three classes of unstable angina severity: class I, characterized as new onset, severe, or accelerated angina; class II, subacute angina at rest characterized by increasing severity, duration, or requirement for nitroglycerin; and class III, characterized as acute angina at rest. Unstable angina represents the clinical state between stable angina and AMI and is thought to be primarily due to the progression in the severity and extent of atherosclerosis, coronary artery spasm, or hemorrhage into non-occluding plaques with subsequent thrombotic occlusion. Coronary angiography of patients with unstable angina usually reveals 90% or greater obstruction of at least one coronary artery, resulting in an inability of oxygen supply to meet even baseline myocardial oxygen demand. Slow growth of stable atherosclerotic plaques or rupture of unstable atherosclerotic plaques with subsequent thrombus formation can cause unstable angina. Both of these causes result in critical narrowing of the coronary artery. Unstable angina is usually associated with atherosclerotic plaque rupture, platelet activation, and thrombus formation. Unstable angina is usually diagnosed by clinical symptoms, ECG changes, and changes in cardiac markers (if any). Treatments for patients with unstable angina include nitrates, aspirin, GPIlb/IIIa inhibitors, heparin, and beta-blockers. Thrombolytic therapy has not been demonstrated to be beneficial for unstable angina patients, and calcium channel blockers may have no effect. Patients may also receive angioplasty and stents. Finally, patients with unstable angina are at risk for developing AMI.

Myocardial infarction is characterized by constricting chest pain lasting longer than 30 minutes that can be accompanied by diagnostic ECG Q waves. Most patients with AMI have coronary artery disease, and as many as 25% of AMI cases are “silent” or asymptomatic infarctions, and individuals with diabetes tend to be more susceptible to silent infarctions. Population studies suggest that 20-60% of nonfatal myocardial infarctions are silent infarctions that are not recognized by the patient. A typical clinical presentations of AMI can include congestive heart failure, angina pectoris without a severe or prolonged attack, a typical location of pain, central nervous system manifestations resembling stroke, apprehension and nervousness, sudden mania or psychosis, syncope, weakness, acute indigestion, and peripheral embolization. AMI is usually diagnosed by clinical symptoms, ECG changes, and elevations of cardiac proteins, most notably cardiac troponin, creatine kinase-MB and myoglobin. Treatments of AMI have improved over the past decade, resulting in improved patient outcome and a 30% decrease in the death rate associated with AMI. Treatment of AMI patients is accomplished by administering agents that limit infarct size and improve outcome by removing occlusive material, increasing the oxygen supply to cardiac tissue, or decreasing the oxygen demand of cardiac tissue. Treatments can include the following: supplemental oxygen, aspirin, GPIIb/IIIa inhibitors, heparin, thrombolytics (tPA), nitrates (nitroglycerin), magnesium, calcium channel antagonists, β-adrenergic receptor blockers, angiotensin-converting enzyme inhibitors, angioplasty (PTCA), and intraluminal coronary artery stents.

The 30 minute time point from chest pain onset is thought to represent the window of reversible myocardial damage caused by ischemia. Stable angina and unstable angina are characterized angiographically as 50-70% and 90% or greater arterial occlusion, respectively, and myocardial infarction is characterized by complete or nearly complete occlusion. A common misconception is that stable angina and unstable angina refer to plaque stability, or that they, along with myocardial infarction, are separate diseases. Because stable angina often progresses to unstable angina, and unstable angina often progresses to myocardial infarction, stable angina, unstable angina, and myocardial infarction can all be characterized as coronary artery disease of varying severity. Recently, the following physiological model of coronary artery disease progression has been proposed: Inflammation→Plaque Rupture→Platelet Activation→Early Thrombosis→Early Necrosis. This model is designed to fit the theory that inflammation occurs during stable angina, and that markers of plaque rupture, platelet activation, and early thrombosis can be used to identify and monitor the progressing severity of unstable angina. The myocardial damage caused during an anginal attack is, by definition, reversible, while damage caused during a myocardial infarction is irreversible. Therefore, there are two proposed break points in this model for the discrimination of stable angina, unstable angina, and AMI. The first occurs between inflammation and plaque rupture, with the theory that plaque rupture does not occur in stable angina. The second occurs between early thrombosis and early necrosis, with the theory that myocardial damage incurred during unstable angina is reversible. It is important to realize that these events, with the exception of early myocardial necrosis, can be associated with all forms of coronary artery disease, and that progression along this diagnostic pathway does not necessarily indicate disease progression. The progression of coronary artery disease from mild unstable angina to severe unstable angina and myocardial infarction is related to plaque instability and the degree of arterial occlusion. This progression can occur slowly, as stable plaques enlarge and become more occlusive, or it can occur rapidly, as unstable plaques rupture, causing platelet activation and occlusive thrombus formation. Because myocardial infarction most frequently shares the same pathophysiology as unstable angina, it is possible that the only distinction between these two events is the reversibility of myocardial damage. By definition, unstable angina causes reversible damage, while myocardial infarction causes irreversible damage. There have been published reports that indicate the presence of myocardial necrosis in patients with unstable angina. By definition, these patients may actually be experiencing early AMI. Nevertheless, even if these patients are diagnosed with unstable angina instead of early AMI, the high degree of severity suggests that they will benefit greatly from early aggressive treatment. Myocardial ischemia is the major determinant in the pathogenesis of stable angina, unstable angina, and myocardial infarction, and they should not be thought of as individual diseases. Rather, they reflect the increasing severity of myocardial damage from ischemia.

Inflammatory mechanisms play a pivotal role in the atherosclerotic process. At the base of atherogenesis there are complex interactions between macrophages, T lymphocytes and smooth muscle cells. A growing body of experimental evidence suggests that inflammation is involved in the pathogenesis of ACS and influences its clinical evolution. In patients with ACS, coronary atherosclerotic plaques are characterized by an abundant inflammatory infiltrate. Moreover, in these patients systemic signs of inflammatory reaction can be observed: activated circulating inflammatory cells (neutrophil, monocytes and lymphocytes) and increased concentrations of pro-inflammatory cytokines, such as interleukin (IL)-1 and 6, and of acute phase reactants, in particular C-reactive protein (CRP).

The Coagulation Cascade in ACS

There are essentially two mechanisms that are used to halt or prevent blood loss following vessel injury. The first mechanism involves the activation of platelets to facilitate adherence to the site of vessel injury. The activated platelets then aggregate to form a platelet plug that reduces or temporarily stops blood loss. The processes of platelet aggregation, plug formation and tissue repair are all accelerated and enhanced by numerous factors secreted by activated platelets. Platelet aggregation and plug formation is mediated by the formation of a fibrinogen bridge between activated platelets. Concurrent activation of the second mechanism, the coagulation cascade, results in the generation of fibrin from fibrinogen and the formation of an insoluble fibrin clot that strengthens the platelet plug.

The coagulation cascade is an enzymatic pathway that involves numerous serine proteinases normally present in an inactive, or zymogen, form. The presence of a foreign surface in the vasculature or vascular injury results in the activation of the intrinsic and extrinsic coagulation pathways, respectively. A final common pathway is then followed, which results in the generation of fibrin by the serine proteinase thrombin and, ultimately, a crosslinked fibrin clot. In the coagulation cascade, one active enzyme is formed initially, which can activate other enzymes that activate others, and this process, if left unregulated, can continue until all coagulation enzymes are activated. Fortunately, there are mechanisms in place, including fibrinolysis and the action of endogenous proteinase inhibitors that can regulate the activity of the coagulation pathway and clot formation.

Fibrinolysis is the process of proteolytic clot dissolution. In a manner analogous to coagulation, fibrinolysis is mediated by serine proteinases that are activated from their zymogen form. The serine proteinase plasmin is responsible for the degradation of fibrin into smaller degradation products that are liberated from the clot, resulting in clot dissolution. Fibrinolysis is activated soon after coagulation in order to regulate clot formation. Endogenous serine proteinase inhibitors also function as regulators of fibrinolysis.

Platelets are round or oval disks with an average diameter of 2-4 μm that are normally found in blood at a concentration of 200,000-300,000 μl. They play an essential role in maintaining hemostasis by maintaining vascular integrity, initially stopping bleeding by forming a platelet plug at the site of vascular injury, and by contributing to the process of fibrin formation to stabilize the platelet plug. When vascular injury occurs, platelets adhere to the site of injury and each other and are stimulated to aggregate by various agents released from adherent platelets and injured endothelial cells. This is followed by the release reaction, in which platelets secrete the contents of their intracellular granules, and formation of the platelet plug. The formation of fibrin by thrombin in the coagulation cascade allows for consolidation of the plug, followed by clot retraction and stabilization of the plug by crosslinked fibrin. Active thrombin, generated in the concurrent coagulation cascade, also has the ability to induce platelet activation and aggregation.

The first step of the common pathway of the coagulation cascade involves the proteolytic cleavage of prothrombin by the factor Xa/factor Va prothrombinase complex to yield active thrombin. Thrombin is a serine proteinase that proteolytically cleaves fibrinogen to form fibrin, which is ultimately integrated into a crosslinked network during clot formation.

The coagulation cascade can be activated through either the extrinsic or intrinsic pathways. These enzymatic pathways share one final common pathway. The first step of the common pathway involves the proteolytic cleavage of prothrombin by the factor Xa/factor Va prothrombinase complex to yield active thrombin. Thrombin is a serine proteinase that proteolytically cleaves fibrinogen.

Thrombus Precursor Protein

Thrombin first removes fibrinopeptide A from fibrinogen, yielding desAA fibrin monomer, which can form complexes with all other fibrinogen-derived proteins, including fibrin degradation products, fibrinogen degradation products, desAA fibrin, and fibrinogen. The desAA fibrin monomer is generically referred to as soluble fibrin, as it is the first product of fibrinogen cleavage, but it is not yet crosslinked via factor XIIIa into an insoluble fibrin clot. DesAA fibrin monomer also can undergo further proteolytic cleavage by thrombin to remove fibrinopeptide B, yielding desAABB fibrin monomer. This monomer can polymerize with other desAABB fibrin monomers to form soluble desAABB fibrin polymer, also referred to as soluble fibrin or thrombus precursor protein (TpPTM).

TpP™ is the immediate precursor to insoluble fibrin, which forms a “mesh-like” structure to provide structural rigidity to the newly formed thrombus. In this regard, measurement of TpP™ in plasma is a direct measurement of active clot formation. The normal plasma concentration of TpP™ was reported to be<6 ng/ml (Laurino, J. P. et al., Ann. Clin. Lab. Sci. 27:338-345, 1997). American Biogenetic Sciences has developed an assay for TpP™ (U.S. Pat. Nos. 5,453,359, 5,837,540 and 5,843,690). Studies have measured elevated TpP™ in patients with AMI (Laurino et al., Ann. Clin. Lab. Sci. 27:338-345, 1997; Carville et al., Clin. Chem. 42:1537-1541, 1996). The plasma concentration of TpP™ is also reported to be elevated in patients with unstable angina (Laurino et al., Ann. Clin. Lab. Sci. 27:338-345, 1997), though other workers have found TpP™ levels to be similar in controls, unstable angina, and chronic stable effort angina (Fiotta et al., Blood Coagul. Fibrinolysis 13: 247-255, 2002).

The concentration of TpP™ in plasma will theoretically be elevated during any condition that causes or is a result of coagulation activation, including disseminated intravascular coagulation, sepsis, pulmonary embolism, deep venous thrombosis, congestive heart failure, surgery, cancer, gastroenteritis, and cocaine overdose (Laurino et al., Ann. Clin. Lab. Sci. 27:338-345, 1997; Song et al., Haematologica 87: 1062-1067, 2002; La Capra et al., Blood Coagul. Fibrinolysis 11: 371-377, 2000). TpP™ is released into the bloodstream immediately following thrombin activation. TpP™ likely has a short half-life in the bloodstream because it will be rapidly converted to insoluble fibrin at the site of clot formation. Plasma TpP™ concentrations are reported to peak within 3 hours of AMI onset, returning to normal after 12 hours from onset. The plasma concentration of TpP™ can exceed 30 ng/ml in CVD (Laurino et al., Ann. Clin. Lab. Sci. 27:338-345, 1997).

MCP-1

Monocyte chemotactic protein-1 (also called monocyte chemoattractant protein-1) (MCP-1) is a 10 kDa chemotactic factor that attracts monocytes and basophils, but not neutrophils or eosiniphils. MCP-1 is normally found in equilibrium between a monomeric and homodimeric form, and it is normally produced in and secreted by monocytes and vascular endothelial cells (Yoshimura, T. et al., FEBS Lett. 244:487-493, 1989; Li, Y. S. et al., Mol. Cell. Biochem. 126:61-68, 1993). MCP-1 has been implicated in the pathogenesis of a variety of diseases that involve monocyte infiltration, including psoriasis, rheumatoid arthritis, and atherosclerosis. The normal concentration of MCP-1 in plasma is <0.1 ng/ml. The plasma concentration of MCP-1 is elevated in patients with AMI, and may be elevated in the plasma of patients with unstable angina, but no elevations have been associated with stable angina (Soejima, H. et al., J. Am. Coll. Cardiol. 34:983-988, 1999; Nishiyama, K. et al., Jpn. Circ. J 62:710-712, 1998; Matsumori, A. et al., J. Mol. Cell. Cardiol. 29:419-423, 1997). Interestingly, MCP-1 also may be involved in the recruitment of monocytes into the arterial wall during atherosclerosis.

Elevations of the serum concentration of MCP-1 are associated with various conditions associated with inflammation, including alcoholic liver disease, interstitial lung disease, sepsis, and systemic lupus erythematosus (Fisher, N. C. et al., Gut 45:416-420, 1999; Suga, M. et al., Eur. Respir. J 14:376-382, 1999; Bossink, A. W. et al., Blood 86:3841-3847, 1995; Kaneko, H. et al. J. Rheumatol. 26:568-573, 1999). MCP-1 is released into the bloodstream upon activation of monocytes and endothelial cells. The concentration of MCP-1 in plasma form patients with AMI has been reported to approach 1 ng/ml (100 pM), and can remain elevated for one month (Soejima, H. et al., J. Am. Coll. Cardiol. 34:983-988, 1999). The kinetics of MCP-1 release into and clearance from the bloodstream in the context of ACS are currently unknown. MCP-1 is a specific marker of the presence of a pro-inflammatory condition that involves monocyte migration.

Definitions

The term “marker” as used herein refers to proteins, polypeptides, phospholipids, or small molecules to be used as targets for screening test samples obtained from subjects. “Proteins or polypeptides” used as markers in the present invention are contemplated to include any fragments thereof, in particular, immunologically detectable fragments.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆. Additionally, related markers may be the result of covalent modification of the parent marker, for example by oxidation of methionine residues, ubiquitination, etc.

The sequence of the 108 amino acid BNP precursor pro-BNP (BNP₁₋₁₀₈) is as follows, with mature BNP (BNP₇₇₋₁₀₈) underlined:

| | | |
|---|---|---|
| HPLGSPGSAS DLETSGLQEQ RNHLQGKLSE LQVEQTSLEP LQESPRPTGV | 50 | (SEQ ID NO: 1) |
| WKSREVATEG IRGHRKMVLY TLRAPRSPKM VQGSGCFGRK MDRISSSSGL | 100 | |
| GCKVLRRH. | 108 | |

BNP₁₋₁₀₈ is synthesized as a larger precursor pre-pro-BNP having the following sequence (with the “pre” sequence shown in bold):

| | | |
|---|---|---|
| MDPQTAPSRA LLLLLFLHLA FLGGRSHPLG SPGSASDLET SGLQEQRNHL | 50 | (SEQ ID NO: 2) |
| QGKLSELQVE QTSLEPLQES PRPTGVWKSR EVATEGIRGH RKMVLYTLRA | 100 | |
| PRSPKMVQGS GCFGRKMDRI SSSSGLGCKVLRRH. | 134 | |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. patent Ser. No. 10/419,059, filed Apr. 17, 2003, which is hereby incorporated by reference in its entirety including all tables, figures and claims.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described above for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (e.g., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returing to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

Preferably, the methods described hereinafter utilize one or more markers that are derived from the subject. The term “subject-derived marker” as used herein refers to protein, polypeptide, phospholipid, nucleic acid, prion, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future stroke in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of delayed neurologic deficits in a patient after stroke, or the chance of future stroke.

The term “correlating,” as used herein in reference to the use of diagnostic and prognostic markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome.

The phrase “determining the diagnosis” as used herein refers to methods by which the skilled artisan can determine the presence or absence of a particular disease in a patient. The term “diagnosis” does not refer to the ability to determine the presence or absence of a particular disease with 100% accuracy, or even that a given course or outcome is more likely to occur than not. Instead, the skilled artisan will understand that the term “diagnosis” refers to an increased probability that a certain disease is present in the subject. In preferred embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

Similarly, the phrase “determining the prognosis” as used herein refers to methods by which the skilled artisan can determine the likelihood of one or more future clinical outcomes for a patient. The skilled artisan will understand that the term “prognosis” refers to an increased probability that a certain clinical outcome will occur at a future date in the subject. In preferred embodiments, a prognosis indicates about a 5% increased chance of a certain clinical outcome compared to a “control” population, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “specific marker of myocardial injury” as used herein refers to molecules that are typically associated with cardiac tissue, and which can be correlated with a cardiac injury, but are not correlated with other types of injury. Such specific markers of cardiac injury include annexin V, B-type natriuretic peptide, β-enolase, cardiac troponin I (free and/or complexed), cardiac troponin T (free and/or complexed), creatine kinase-MB, glycogen phosphorylase-BB, heart-type fatty acid binding protein, phosphoglyceric acid mutase-MB, and S-100ao. These.

The term “specific marker of neural tissue injury” as used herein refers to molecules that are typically associated with neural tissue, and which can be correlated with a neural injury, but are not correlated with other types of injury. Exemplary specific markers of neural tissue injury are described in detail hereinafter.

Exemplary Markers for Use in Panels with TpP™ and/or MCP-1

(i) Specific Markers of Myocardial Injury

The following are exemplary specific markers of myocardial injury. This list is not meant to be limiting.

Annexin V, also called lipocortin V, endonexin II, calphobindin I, calcium binding protein 33, placental anticoagulant protein I, thromboplastin inhibitor, vascular anticoagulant-α, and anchorin CII, is a 33 kDa calcium-binding protein that is an indirect inhibitor and regulator of tissue factor. Giambanco, I. et al., J. Histochem. Cytochem. 39:P1189-1198, 1991; Doubell, A. F. et al., Cardiovasc. Res. 27:1359-1367, 1993. The normal plasma concentration of annexin V is <2 ng/ml (Kaneko, N. et al., Clin. Chim. Acta 251:65-80, 1996). One study has found that the plasma concentration of annexin V is elevated in individuals with AMI, but not significantly elevated in patients with old myocardial infarction, chest pain syndrome, valvular heart disease, lung disease, and kidney disease. Kaneko, N. et al. Clin. Chim. Acta 251:65-80, 1996.

Enolase is a 78 kDa homo- or heterodimeric cytosolic protein produced from α, β, and γ subunits. Enolase catalyzes the interconversion of 2-phosphoglycerate and phosphoenolpyruvate in the glycolytic pathway. Enolase is present as αα, αβ, ββ, αγ, and γγ isoforms. The α subunit is found in most tissues, the β subunit is found in cardiac and skeletal muscle, and the γ subunit is found primarily in neuronal and neuroendocrine tissues. β-enolase is composed of αβ and ββ enolase, and is specific for muscle. β-enolase is reported to be elevated in the serum of individuals with AMI, but not in individuals with angina (Nomura, M. et al., Br. Heart J. 58:29-33, 1987; Herraez-Dominguez, M. V. et al., Clin. Chim. Acta 64:307-315, 1975). The plasma concentration of β-enolase is also elevated during heart surgery, muscular dystrophy, and skeletal muscle injury (Usui, A. et al., Cardiovasc. Res. 23:737-740, 1989; Kato, K. et al., Clin. Chim. Acta 131:75-85, 1983; Matsuda, H. et al., Forensic Sci. Int. 99:197-208, 1999).

Troponin I (TnI) is a 25 kDa inhibitory element of the troponin complex, found in all striated muscle tissue. TnI binds to actin in the absence of Ca²⁺, inhibiting the ATPase activity of actomyosin. A TnI isoform that is found in cardiac tissue (cTnI) is 40% divergent from skeletal muscle TnI, allowing both isoforms to be immunologically distinguished. The plasma cTnI concentration may be elevated in conjunction with cardiac trauma, congestive heart failure, and cardiac surgery, non-ischemic dilated cardiomyopathy, muscular disorders, CNS disorders, HIV infection, chronic renal failure, sepsis, lung disease, and endocrine disorders (Khan, I. A. et al., Am. J. Emerg. Med. 17:225-229, 1999). cTnI is released into the bloodstream following cardiac cell death. The plasma concentration of cTnI in patients with AMI is significantly elevated 4-6 hours after onset, peaks between 12-16 hours, and can remain elevated for one week. The release kinetics of cTnI associated with unstable angina may be similar. The measurement of specific forms of cardiac troponin, including free cardiac troponin I and complexes of cardiac troponin I with troponin C and/or T may provide the user with the ability to identify various stages of ACS.

Free and complexed cardiac-troponin T may be used in a manner analogous to that described above for cardiac troponin I. Cardiac troponin T complex may be useful either alone or when expressed as a ratio with total cardiac troponin I to provide information related to the presence of progressing myocardial damage. Ongoing ischemia may result in the release of the cardiac troponin TIC complex, indicating that higher ratios of cardiac troponin TIC:total cardiac troponin I may be indicative of continual damage caused by unresolved ischemia.

Creatine kinase (CK) is an 85 kDa cytosolic enzyme that catalyzes the reversible formation ADP and phosphocreatine from ATP and creatine. CK is a homo- or heterodimer composed of M and B chains. CK-MB is the isoform that is most specific for cardiac tissue, but it is also present in skeletal muscle and other tissues. Plasma CK-MB concentrations are significantly elevated in patients with AMI. Thygesen, K. et al., Eur. J. Clin. Invest. 16:1-4, 1986; Koukkunen, H. et al., Ann. Med. 30:488-496, 1998; Bertinchant, J. P. et al., Clin. Biochem. 29:587-594, 1996; Benamer, H. et al., Am. J. Cardiol. 82:845-850, 1998; Norregaard-Hansen, K. et al., Eur. Heart J. 13:188-193, 1992. CK-MB may be useful in determining the severity of unstable angina because the extent of myocardial ischemia is directly proportional to unstable angina severity. Elevations of the plasma CK-MB concentration are associated with skeletal muscle injury and renal disease.

Glycogen phosphorylase (GP) is a 188 kDa intracellular allosteric enzyme that catalyzes the removal of glucose (liberated as glucose-1-phosphate) from the nonreducing ends of glycogen in the presence of inorganic phosphate during glycogenolysis. GP is present as a homodimer, which associates with another homodimer to form a tetrameric enzymatically active phosphorylase A. There are three isoforms of GP that can be immunologically distinguished. The BB isoform is found in brain and cardiac tissue, the MM isoform is found in skeletal muscle and cardiac tissue, and the LL isoform is predominantly found in liver (Mair, J. et al., Br. Heart J. 72:125-127, 1994). The plasma GP-BB concentration is significantly elevated in patients with AMI and unstable angina with transient ST-T elevations, but not stable angina (Mair, J. et al., Br. Heart J. 72:125-127, 1994; Mair, J., Clin. Chim. Acta 272:79-86, 1998; Rabitzsch, G. et al., Clin. Chem. 41:966-978, 1995; Rabitzsch, G. et al., Lancet 341:1032-1033, 1993). GP-BB also can be used to detect perioperative AMI and myocardial ischemia in patients undergoing coronary artery bypass surgery (Rabitzsch, G. et al., Biomed. Biochim. Acta 46:S584-S588, 1987; Mair, P. et al., Eur. J. Clin. Chem. Clin. Biochem. 32:543-547, 1994). Because it is also found in the brain, the plasma GP-BB concentration also may be elevated during ischemic cerebral injury.

Heart-type fatty acid binding protein (H-FABP) is a cytosolic 15 kDa lipid-binding protein involved in lipid metabolism. Heart-type FABP antigen is found not only in heart tissue, but also in kidney, skeletal muscle, aorta, adrenals, placenta, and brain (Veerkamp, J. H. and Maatman, R. G., Prog. Lipid Res. 34:17-52, 1995; Yoshimoto, K. et al., Heart Vessels 10:304-309, 1995). The plasma H-FABP concentration is elevated in patients with AMI and unstable angina (Ishii, J. et al., Clin. Chem. 43:1372-1378, 1997; Tsuji, R. et al., Int. J. Cardiol. 41:209-217, 1993). Myocardial tissue as a source of H-FABP can be confirmed by determining the ratio of myoglobin/FABP (grams/grams). Van Nieuwenhoven, F. A. et al., Circulation 92:2848-2854, 1995. The plasma H-FABP concentration can be significantly elevated 1-2 hours after the onset of chest pain, earlier than CK-MB and myoglobin (Tsuji, R. et al., Int. J. Cardiol. 41:209-217, 1993; Van Nieuwenhoven, F. A. et al., Circulation 92:2848-2854, 1995; Tanaka, T. et al., Clin. Biochem. 24:195-201, 1991).

Phosphoglyceric acid mutase (PGAM) is a 57 kDa homo- or heterodimeric intracellular glycolytic enzyme composed of 29 kDa M or B subunits that catalyzes the interconversion of 3-phosphoglycerate to 2-phosphoglycerate in the presence of magnesium. Cardiac tissue contains isozymes MM, MB, and BB, while skeletal muscle contains primarily PGAM-MM, and most other tissues contain PGAM-BB (Durany, N. and Carreras, J., Comp. Biochem. Physiol. B. Biochem. Mol. Biol. 114:217-223, 1996).

S-100 is a 21 kDa homo- or heterodimeric cytosolic Ca²⁺-binding protein produced from α and β subunits. It is thought to participate in the activation of cellular processes along the Ca²⁺-dependent signal transduction pathway (Bonfrer, J. M. et al., Br. J. Cancer 77:2210-2214, 1998). S-100ao (αα isoform) is found in striated muscles, heart and kidney, S-100a (αβ isoform) is found in glial cells, but not in Schwann cells, and S-100β (ββ isoform) is found in high concentrations in glial cells and Schwann cells, where it is a major cytosolic component (Kato, K. and Kimura, S., Biochim. Biophys. Acta 842:146-150, 1985; Hasegawa, S. et al., Eur. Urol. 24:393-396, 1993). The serum concentration of S-100ao was reported to be elevated in patients with AMI, but not in patients with angina pectoris with suspected AMI (Usui, A. et al., Clin. Chem. 36:639-641, 1990). The serum concentration of S-100ao is significantly elevated on admission in patients with AMI, increases to peak levels 8 hours after admission, decreases and returns to baseline one week later (Usui, A. et al., Clin. Chem. 36:639-641, 1990). Furthermore, S-100ao appears to be significantly elevated earlier after AMI onset than CK-MB (Usui, A. et al., Clin. Chem. 36:639-641, 1990).

(ii) Exemplary Markers Related to Blood Pressure Regulation

In addition to BNP and markers related thereto, discussed in detail above, the following represent exemplary markers that are known in the art to be related to blood pressure regulation. This list is not meant to be limiting.

A-type natriuretic peptide (ANP) (also referred to as atrial natriuretic peptide or cardiodilatin (Forssmann et al Histochem Cell Biol 110: 335-357, 1998) is a 28 amino acid peptide that is synthesized, stored, and released atrial myocytes in response to atrial distension, angiotensin II stimulation, endothelin, and sympathetic stimulation (beta-adrenoceptor mediated). ANP is synthesized as a precursor molecule (pro-ANP) that is converted to an active form, ANP, by proteolytic cleavage and also forming N-terminal ANP (1-98). N-terminal ANP and ANP have been reported to increase in patients exhibiting atrial fibrillation and heart failure (Rossi et al. Journal of the American College of Cardiology 35: 1256-62, 2000). In addition to atrial natriuretic peptide (ANP99-126) itself, linear peptide fragments from its N-terminal prohormone segment have also been reported to have biological activity. As the skilled artisan will recognize, however, because of its relationship to ANP, the concentration of N-terminal ANP molecule can also provide diagnostic or prognostic information in patients. The phrase “marker related to ANP or ANP related peptide” refers to any polypeptide that originates from the pro-ANP molecule (1-126), other than the 28-amino acid ANP molecule itself. Proteolytic degradation of ANP and of peptides related to ANP have also been described in the literature and these proteolytic fragments are also encompassed it the term “ANP related peptides.”

C-type natriuretic peptide (CNP) is a 22-amino acid peptide that is the primary active natriuretic peptide in the human brain; CNP is also considered to be an endothelium-derived relaxant factor, which acts in the same way as nitric oxide (NO) (Davidson et al., Circulation 93:1155-9, 1996). CNP is structurally related to Atrial natriuretic peptide (ANP) and B-type natriuretic peptide (BNP); however, while ANP and BNP are synthesized predominantly in the myocardium, CNP is synthesized in the vascular endothelium as a precursor (pro-CNP) (Prickett et al., Biochem. Biophys. Res. Commun. 286:513-7, 2001).

Urotensin II is a peptide having the sequence Ala-Gly-Thr-Ala-Asp-Cys-Phe-Trp-Lys-Tyr-Cys-Val, with a disulfide bridge between Cys6 and Cys 11. Human urotensin 2 (UTN) is synthesized in a prepro form. Processed urotensin 2 has potent vasoactive and cardiostimulatory effects, acting on the G protein-linked receptor GPR14.

Vasopressin (arginine vasopressin, AVP; antidiuretic hormone, ADH) is a peptide hormone released from the posterior pituitary. Its primary function in the body is to regulate extracellular fluid volume by affecting renal handling of water. There are several mechanisms regulating release of AVP. Hypovolemia, as occurs during hemorrhage, results in a decrease in atrial pressure. Specialized stretch receptors within the atrial walls and large veins (cardiopulmonary baroreceptors) entering the atria decrease their firing rate when there is a fall in atrial pressure. Afferent from these receptors synapse within the hypothalamus; atrial receptor firing normally inhibits the release of AVP by the posterior pituitary. With hypovolemia or decreased central venous pressure, the decreased firing of atrial stretch receptors leads to an increase in AVP release. Hypothalamic osmoreceptors sense extracellular osmolarity and stimulate AVP release when osmolarity rises, as occurs with dehydration. Finally, angiotensin II receptors located in a region of the hypothalamus regulate AVP release—an increase in angiotensin II simulates AVP release.

Calcitonin gene related peptide (CGRP) is a polypeptide of 37 amino acids that is a product of the calcitonin gene derived by alternative splicing of the precursor mRNA. The calcitonin gene (CALC-I) primary RNA transcript is processed into different mRNA segments by inclusion or exclusion of different exons as part of the primary transcript. Calcitonin-encoding mRNA is the main product of CALC-I transcription in C-cells of the thyroid, whereas CGRP-I mRNA (CGRP=calcitonin-gene-related peptide) is produced in nervous tissue of the central and peripheral nervous systems. In the third mRNA sequence, the calcitonin sequence is lost and alternatively the sequence of CGRP is encoded in the mRNA. CGRP is a markedly vasoactive peptide with vasodilatative properties. CGRP has no effect on calcium and phosphate metabolism and is synthesised predominantly in nerve cells related to smooth muscle cells of the blood vessels. ProCGRP, the precursor of CGRP, and PCT have partly identical N-terminal amino acid sequences.

Procalcitonin is a 116 amino acid (14.5 kDa) protein encoded by the Calc-1 gene located on chromosome 11p15.4. The Calc-1 gene produces two transcripts that are the result of alternative splicing events. Pre-procalcitonin contains a 25 amino acid signal peptide which is processed by C-cells in the thyroid to a 57 amino acid N-terminal fragment, a 32 amino acid calcitonin fragment, and a 21 amino acid katacalcin fragment. Procalcitonin is secreted intact as a glycosylated product by other body cells. Whicher et al., Ann. Clin. Biochem. 38: 483-93 (2001). Plasma procalcitonin has been identified as a marker of sepsis and its severity. Yukioka et al., Ann. Acad. Med. Singapore 30: 528-31 (2001); Pettila et al., Intensive Care Med. 28: 1220-25 (2002).

Angiotensin II is an octapeptide hormone formed by renin action upon a circulating substrate, angiotensinogen, that undergoes proteolytic cleavage to from the decapeptide angiotensin I. Vascular endothelium, particularly in the lungs, has an enzyme, angiotensin converting enzyme (ACE), that cleaves off two amino acids to form the octapeptide, angiotensin II (AII).

Adrenomedullin (AM) is a 52-amino acid peptide which is produced in many tissues, including adrenal medulla, lung, kidney and heart (Yoshitomi et al., Clin. Sci. (Colch) 94:135-9, 1998). Intravenous administration of AM causes a long-lasting hypotensive effect, accompanied with an increase in the cardiac output in experimental animals. AM is synthesized as a precursor molecule (pro-AM). The N-terminal peptide processed from the AM precursor has also been reported to act as a hypotensive peptide (Kuwasako et al., Ann. Clin. Biochem. 36:622-8, 1999).

The endothelins are three related peptides (endothelin-1, endothelin-2, and endothelin-3) encoded by separate genes that are produced by vascular endothelium, each of which exhibit potent vasoconstricting activity. Endothelin-1 (ET-1) is a 21 amino acid residue peptide, synthesized as a 212 residue precursor (preproET-1), which contains a 17 residue signal sequence that is removed to provide a peptide known as big ET-1. This molecule is further processed by hydrolysis between trp21 and val22 by endothelin converting enzyme. Both big ET-1 and ET-1 exhibit biological activity; however the mature ET-1 form exhibits greater vasoconstricting activity (Brooks and Ergul, J. Mol. Endocrinol. 21:307-15, 1998). Similarly, endothelin-2 and endothelin-3 are also 21 amino acid residues in length, and are produced by hydrolysis of big endothelin-2 and big endothelin-3, respectively (Yap et al., Br. J. Pharmacol. 129:170-6, 2000; Lee et al., Blood 94:1440-50, 1999).

(ii) Exemplary Markers Related to Coagulation and Hemostasis

Elevations in the serum concentration of markers related to coagulation and hemostasis may be associated with clot presence, or any condition that causes or is a result of fibrinolysis activation, including atherosclerosis, disseminated intravascular coagulation, acute myocardial infarction, surgery, trauma, unstable angina, stroke, pulmonary embolsim, venous thrombosis, and thrombotic thrombocytopenic purpura. The following are exemplary markers related to coagulation and hemostasis. This list is not meant to be limiting.

D-dimer is a crosslinked fibrin degradation product with an approximate molecular mass of 200 kDa. The plasma concentration of D-dimer is elevated in patients with acute myocardial infarction and unstable angina, but not stable angina. Hoffineister, H. M. et al., Circulation 91: 2520-27 (1995); Bayes-Genis, A. et al., Thromb. Haemost. 81: 865-68 (1999); Gurfinkel, E. et al., Br. Heart J 71: 151-55 (1994); Kruskal, J. B. et al., N. Engl. J. Med. 317: 1361-65 (1987); Tanaka, M. and Suzuki, A., Thromb. Res. 76: 289-98 (1994). D-dimer is released into the bloodstream immediately following proteolytic clot dissolution by plasmin. The plasma concentration of D-dimer is also reported to be elevated in patients with acute pulmonary embolism. Egermayer et al., Thorax 53: 830-34 (1998).

Plasmin is a 78 kDa serine proteinase that proteolytically digests crosslinked fibrin, resulting in clot dissolution. The 70 kDa serine proteinase inhibitor α2-antiplasmin (α2AP) regulates plasmin activity by forming a covalent 1:1 stoichiometric complex with plasmin. The resulting ˜150 kDa plasminαa2AP complex (PAP), also called plasmin inhibitory complex (PIC) is formed immediately after α2AP comes in contact with plasmin that is activated during fibrinolysis.

β-thromboglobulin (βTG) is a 36 kDa platelet α granule component that is released upon platelet activation. Plasma levels of β-TG appear to be elevated in patients with unstable angina and acute myocardial infarction, but not stable angina (De Caterina, R. et al., Eur. Heart J. 9:913-922, 1988; Bazzan, M. et al., Cardiologia 34, 217-220, 1989). Plasma β-TG elevations also seem to be correlated with episodes of ischemia in patients with unstable angina (Sobel, M. et al., Circulation 63:300-306, 1981). Plasma concentrations of βTG associated with ACS can approach 70 ng/ml (2 nM), but this value may be influenced by platelet activation during the sampling procedure.

Platelet factor 4 (PF4) is a 40 kDa platelet a granule component that is released upon platelet activation. PF4 is a marker of platelet activation and has the ability to bind and neutralize heparin. The plasma concentration of PF4 appears to be elevated in patients with acute myocardial infarction and unstable angina, but not stable angina (Gallino, A. et al., Am. Heart J. 112:285-290, 1986; Sakata, K. et al., Jpn. Circ. J. 60:277-284, 1996; Bazzan, M. et al., Cardiologia 34:217-220, 1989). Plasma PF4 elevations also seem to be correlated with episodes of ischemia in patients with unstable angina (Sobel, M. et al., Circulation 63:300-306, 1981).

Fibrinopeptide A (FPA) is a 16 amino acid, 1.5 kDa peptide that is liberated from amino terminus of fibrinogen by the action of thrombin. The plasma FPA concentration is elevated in patients with acute myocardial infarction, unstable angina, and variant angina, but not stable angina (Gensini, G. F. et al., Thromb. Res. 50:517-525, 1988; Gallino, A. et al., Am. Heart J 112:285-290, 1986; Sakata, K. et al., Jpn. Circ. J. 60:277-284, 1996; Theroux, P. et al., Circulation 75:156-162, 1987; Merlini, P. A. et al., Circulation 90:61-68, 1994; Manten, A. et al., Cardiovasc. Res. 40:389-395, 1998). Furthermore, plasma FPA may indicate the severity of angina (Gensini, G. F. et al., Thromb. Res. 50:517-525, 1988).

Platelet-derived growth factor (PDGF) is a 28 kDa secreted homo- or heterodimeric protein composed of the homologous subunits A and/or B (Mahadevan, D. et al., J. Biol. Chem. 270:27595-27600, 1995). PDGF is released by aggregating platelets and monocytes near sites of vascular injur, and has been implicated in the pathogenesis of atherosclerosis. Plasma PDGF concentrations are higher in individuals with acute myocardial infarction and unstable angina than in healthy controls or individuals with stable angina (Ogawa, H. et al., Am. J. Cardiol. 69:453-456, 1992; Wallace, J. M. et al., Ann. Clin. Biochem. 35:236-241, 1998; Ogawa, H. et al., Coron. Artery Dis. 4:437-442, 1993).

Prothrombin fragment 1+2 is a 32 kDa polypeptide that is liberated from the amino terminus of thrombin during thrombin activation. The plasma concentration of F1+2 is reportedly elevated in patients with acute myocardial infarction and unstable angina, but not stable angina (Merlini, P. A. et al., Circulation 90:61-68, 1994). Other reports have indicated that there is no significant change in the plasma F1+2 concentration in cardiovascular disease (Biasucci, L. M. et al., Circulation 93:2121-2127, 1996; Manten, A. et al., Cardiovasc. Res. 40:389-395, 1998).

P-selectin, also called granule membrane protein-140, GMP-140, PADGEM, and CD-62P, is a ˜140 kDa adhesion molecule expressed in platelets and endothelial cells. P-selectin is stored in the alpha granules of platelets and in the Weibel-Palade bodies of endothelial cells. Membrane-bound and soluble forms of P-selectin have been identified. Soluble P-selectin may play an important role in regulating inflammation and thrombosis by blocking interactions between leukocytes and activated platelets and endothelial cells (Gamble, J. R. et al., Science 249:414-417, 1990). The plasma soluble P-selectin concentration was significantly elevated in patients with acute myocardial infarction and unstable angina, but not stable angina, even following an exercise stress test (Ikeda, H. et al., Circulation 92:1693-1696, 1995; Tomoda, H. and Aoki, N., Angiology 49:807-813, 1998; Hollander, J. E. et al., J. Am. Coll. Cardiol. 34:95-105, 1999; Kaikita, K. et al., Circulation 92:1726-1730, 1995; Ikeda, H. et al., Coron. Artery Dis. 5:515-518, 1994). The sensitivity and specificity of membrane-bound P-selectin versus soluble P-selectin for acute myocardial infarction is 71% versus 76% and 32% versus 45% (Hollander, J. E. et al., J. Am. Coll. Cardiol. 34:95-105, 1999). The sensitivity and specificity of membrane-bound P-selectin versus soluble P-selectin for unstable angina+acute myocardial infarction is 71% versus 79% and 30% versus 35% (Hollander, J. E. et al., J. Am. Coll. Cardiol. 34:95-105, 1999).

Thrombin is a 37 kDa serine proteinase that proteolytically cleaves fibrinogen to form fibrin, which is ultimately integrated into a crosslinked network during clot formation. Antithrombin III (ATIII) is a 65 kDa serine proteinase inhibitor that is a physiological regulator of thrombin, factor XIa, factor XIIa, and factor IXa proteolytic activity. The normal plasma concentration of the approximately 100 kDa thrombin-ATIII complex (TAT) is <5 ng/ml (50 pM). TAT concentration is elevated in patients with acute myocardial infarction and unstable angina, especially during spontaneous ischemic episodes (Biasucci, L. M. et al., Am. J. Cardiol. 77:85-87, 1996; Kienast, J. et al., Thromb. Haemost. 70:550-553, 1993). Furthermore, TAT may be elevated in the plasma of individuals with stable angina (Manten, A. et al., Cardiovasc. Res. 40:389-395, 1998). Other published reports have found no significant differences in the concentration of TAT in the plasma of patients with ACS (Manten, A. et al., Cardiovasc. Res. 40:389-395, 1998; Hoffineister, H. M. et al., Atherosclerosis 144:151-157, 1999).

von Willebrand factor (vWF) is a plasma protein produced by platelets, megakaryocytes, and endothelial cells composed of 220 kDa monomers that associate to form a series of high molecular weight multimers. These multimers normally range in molecular weight from 600-20,000 kDa. The A1 domain of vWF binds to the platelet glycoprotein Ib-IX-V complex and non-fibrillar collagen type VI, and the A3 domain binds fibrillar collagen types I and III (Emsley, J. et al., J. Biol. Chem. 273:10396-10401, 1998). Other domains present in the vWF molecule include the integrin binding domain, which mediates platelet-platelet interactions, the the protease cleavage domain, which appears to be relevant to the pathogenesis of type 11A von Willebrand disease. Measurement of the total amount of vWF would allow one who is skilled in the art to identify changes in total vWF concentration. This measurement could be performed through the measurement of various forms of the vWF molecule. Measurement of the A1 domain would allow the measurement of active vWF in the circulation, indicating that a pro-coagulant state exists because the A1 domain is accessible for platelet binding. In this regard, an assay that specifically measures vWF molecules with both the exposed A1 domain and either the integrin binding domain or the A3 domain would also allow for the identification of active vWF that would be available for mediating platelet-platelet interactions or mediate crosslinking of platelets to vascular subendothelium, respectively.

Tissue factor (TF) is a 45 kDa cell surface protein expressed in brain, kidney, and heart, and in a transcriptionally regulated manner on perivascular cells and monocytes. Tissue factor can be detected in the bloodstream in a soluble form, bound to factor VIIa, or in a complex with factor VIIa, and tissue factor pathway inhibitor that can also include factor Xa. TF also is expressed on the surface of macrophages, which are commonly found in atherosclerotic plaques. TF is elevated in patients with unstable angina and acute myocardial infarction, but not in patients with stable angina (Falciani, M. et al., Thromb. Haemost. 79:495-499, 1998; Suefuji, H. et al., Am. Heart J. 134:253-259, 1997; Misumi, K. et al., Am. J. Cardiol. 81:22-26, 1998). Furthermore, TF expression on macrophages and TF activity in atherosclerotic plaques is more common in unstable angina than stable angina (Soejima, H. et al., Circulation 99:2908-2913, 1999; Kaikita, K. et al., Arterioscler. Thromb. Vasc. Biol. 17:2232-2237, 1997; Ardissino, D. et al., Lancet 349:769-771, 1997).

(iii) Exemplary Markers Related to the Acute Phase Response

Acute phase proteins are a group of proteins, such as C-reactive protein and mannose-binding protein, produced by cells in the liver and that promote inflammation, activate the complement cascade, and stimulate chemotaxis of phagocytes. The following are exemplary markers related to the acute phase response. This list is not meant to be limiting.

Human neutrophil elastase (HNE) is a 30 kDa serine proteinase that is normally contained within the azurophilic granules of neutrophils. HNE is released upon neutrophil activation, and its activity is regulated by circulating α₁-proteinase inhibitor. The plasma HNE concentration is usually measured by detecting HNE-α₁-PI complexes. The normal concentration of these complexes is 50 ng/ml, which indicates a normal concentration of approximately 25 ng/ml (0.8 nM) for HNE. HNE release also can be measured through the specific detection of fibrinopeptide Bβ₃₀₋₄₃, a specific HNE-derived fibrinopeptide, in plasma. Plasma HNE is elevated in patients with coronary stenosis, and its elevation is greater in patients with complex plaques than those with simple plaques (Kosar, F. et al., Angiology 49:193-201, 1998; Amaro, A. et al., Eur. Heart J. 16:615-622, 1995). Plasma HNE is not significantly elevated in patients with stable angina, but is elevated inpatients with unstable angina and acute myocardial infarction, as determined by measuring fibrinopeptide Bβ₃₀₋₄₃, with concentrations in unstable angina being 2.5-fold higher than those associated with acute myocardial infarction (Dinerman, J. L. et al., J. Am. Coll. Cardiol. 15:1559-1563, 1990; Mehta, J. et al., Circulation 79:549-556, 1989).

Inducible nitric oxide synthase (iNOS) is a 130 kDa cytosolic protein in epithelial cells macrophages whose expression is regulated by cytokines, including interferon-γ, interleukin-1β, interleukin-6, and tumor necrosis factor α, and lipopolysaccharide. iNOS catalyzes the synthesis of nitric oxide (NO) from L-arginine, and its induction results in a sustained high-output production of NO, which has antimicrobial activity and is a mediator of a variety of physiological and inflammatory events. NO production by iNOS is approximately 100 fold more than the amount produced by constitutively-expressed NOS (Depre, C. et al., Cardiovasc. Res. 41:465-472, 1999). iNOS expression during myocardial ischemia may not be elevated, suggesting that iNOS may be useful in the differentiation of angina from acute myocardial infarction (Hammerman, S. I. et al., Am. J. Physiol. 277:H1579-H1592, 1999; Kaye, D. M. et al., Life Sci 62:883-887, 1998).

Lysophosphatidic acid (LPA) is a lysophospholipid intermediate formed in the synthesis of phosphoglycerides and triacylglycerols. In the context of unstable angina, LPA is most likely released as a direct result of plaque rupture.

Malondialdehyde-modified low-density lipoprotein (MDA-modified LDL) is formed during the oxidation of the apoB-100 moiety of LDL as a result of phospholipase activity, prostaglandin synthesis, or platelet activation. Plasma concentrations of oxidized LDL are elevated in stable angina, unstable angina, and acute myocardial infarction, indicating that it may be a marker of atherosclerosis (Holvoet, P., Acta Cardiol. 53:253-260, 1998; Holvoet, P. et al., Circulation 98:1487-1494, 1998). Plasma MDA-modified LDL is not elevated in stable angina, but is significantly elevated in unstable angina and acute myocardial infarction (Holvoet, P., Acta Cardiol. 53:253-260, 1998; Holvoet, P. et al., Circulation 98:1487-1494, 1998; Holvoet, P. et al., JAMA 281:1718-1721, 1999). Plasma concentrations of MDA-modified LDL can approach 20 μg/ml (˜50 μM) in patients with acute myocardial infarction, and 15 μg/ml (˜40 μM) in patients with unstable angina (Holvoet, P. et al., Circulation 98:1487-1494, 1998).

Matrix metalloproteinase-1 (MMP-1), also called collagenase-1, is a 41/44 kDa zinc- and calcium-binding proteinase that cleaves primarily type I collagen, but can also cleave collagen types II, III, VII and X. The active 41/44 kDa enzyme can undergo autolysis to the still active 22/27 kDa form. MMP-1 can be found in the bloodstream either in a free form or in complex with TIMP-1, its natural inhibitor. MMP-1 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Furthermore, MMP-1 has been implicated in the pathogenesis of myocardial reperfusion injury (Shibata, M. et al., Angiology 50:573-582, 1999).

Lipopolysaccharide binding protein (LBP) is a ˜60 kDa acute phase protein produced by the liver. LBP binds to lipopolysaccharide and is involved in LPS handling in humans. LBP has been reported to mediate transfer of LPS to the LPS receptor (CD14) on mononuclear cells, and into HDL. LBP has also been reported to protect mice from septic shock caused by LPS.

Matrix metalloproteinase-2 (MMP-2), also called gelatinase A, is a 66 kDa zinc- and calcium-binding proteinase that is synthesized as an inactive 72 kDa precursor. Mature MMP-3 cleaves type I gelatin and collagen of types IV, V, VII, and X. MMP-2 is usually found in plasma in complex with TIMP-2, its physiological regulator (Murawaki, Y. et al., J. Hepatol. 30:1090-1098, 1999). MMP-2 expression is elevated in vascular smooth muscle cells within atherosclerotic lesions, and it may be released into the bloodstream in cases of plaque instability (Kai, H. et al., J. Am. Coll. Cardiol. 32:368-372, 1998). Serum MMP-2 concentrations were elevated in patients with stable angina, unstable angina, and acute myocardial infarction, with elevations being significantly greater in unstable angina and acute myocardial infarction than in stable angina (Kai, H. et al., J. Am. Coll. Cardiol. 32:368-372, 1998). MMP-2 was elevated on admission in the serum of individuals with unstable angina and acute myocardial infarction, with maximum levels approaching 1.5 μg/ml (25 nM) (Kai, H. et al., J. Am. Coll. Cardiol. 32:368-372, 1998).

Matrix metalloproteinase-3 (MMP-3), also called stromelysin-1, is a 45 kDa zinc- and calcium-binding proteinase that is synthesized as an inactive 60 kDa precursor. The serum MMP-3 concentration in males is approximately 2 times higher than in females (Manicourt, D. H. et al., Arthritis Rheum. 37:1774-1783, 1994). MMP-3 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998

Matrix metalloproteinase-9 (MMP-9) also called gelatinase B, is an 84 kDa zinc- and calcium-binding proteinase that is synthesized as an inactive 92 kDa precursor. MMP-9 exists as a monomer, a homodimer, and a heterodimer with a 25 kDa α₂-microglobulin-related protein (Triebel, S. et al., FEBS Lett. 314:386-388, 1992). Plasma MMP-9 concentrations are significantly elevated in patients with unstable angina and acute myocardial infarction, but not stable angina (Kai, H. et al., J. Am. Coll. Cardiol. 32:368-372, 1998).

The balance between matrix metalloproteinases and their inhibitors is a critical factor that affects tumor invasion and metastasis. The TIMP family represents a class of small (21-28 kDa) related proteins that inhibit the metalloproteinases. Tissue inhibitor of metalloproteinase 1 (TIMP1) is reportedly involved in the regulation of bone modeling and remodeling in normal developing human bone, involved in the invasive phenotype of acute myelogenous leukemia, demonstrating polymorphic X-chromosome inactivation. TIMP 1 is known to act on mmp-1, mmp-2, mmp-3, mmp-7, mmp-8, mmp-9, mmp-10, mmp-11, mmp-12, mmp-13 and mmp-16. Tissue inhibitor of metalloproteinase 2 (TIMP2) complexes with metalloproteinases (such as collagenases) and irreversibly inactivates them. TIMP 2 is known to act on mmp-1, mmp-2, mmp-3, mmp-7, mmp-8, mmp-9, mmp-10, mmp-13, mmp-14, mmp-15, mmp-16 and mmp-19. Two alternatively spliced forms may be associated with SYN4, and involved in the invasive phenotype of acute myelogenous leukemia. Unlike the inducible expression of some other TIMP gene family members, the expression of this gene is largely constitutive. Tissue inhibitor of metalloproteinase 3 (TIMP3) antagonizes matrix metalloproteinase activity and can suppress tumor growth, angiogenesis, invasion, and metastasis. Loss of TIMP-3 has been related to the acquisition of tumorigenesis.

(iv) Exemplary Markers Related to Inflammation

Acute phase proteins are part of a larger group of proteins that are related to local or systemic inflammation. The following exemplary list of additional markers related to inflammation is not meant to be limiting.

Interleukins (ILs) are part of a larger class of polypeptides known as cytokines. These are messenger molecules that transmit signals between various cells of the immune system. They are mostly secreted by macrophages and lymphocytes and their production is induced in response to injury or infection. Their actions influence other cells of the immune system as well as other tissues and organs including the liver and brain. There are at least 18 ILs described. IL-1P, IL-2, IL-4, IL-6, IL-8 and IL-10 are preferred for use as markers in the present invention. The following table shows selected functions of representative interleukins.

TABLE 1
Selected Functions of Representative Interleukins*

| Functions | IL-1 | IL-2 | IL-4 | IL-6 | IL-8 | IL-10 |
|---|---|---|---|---|---|---|
| Enhance immune responses | + | + | + | + | − | + |
| Suppress immune responses | − | − | − | − | − | + |
| Enhance inflammation | + | + | + | + | + | − |
| Suppress inflammation | − | − | − | − | − | + |
| Promote cell growth | + | + | − | − | − | − |
| Chemotactic (chemokines) | − | − | − | − | + | − |
| Pyrogenic | + | − | − | − | − | − |

Interleukin-1β (IL-1β) is a 17 kDa secreted proinflammatory cytokine that is involved in the acute phase response and is a pathogenic mediator of many diseases. IL-1β is normally produced by macrophages and epithelial cells. IL-1β is also released from cells undergoing apoptosis. Elevations of the plasma IL-1β concentration are associated with activation of the acute phase response in proinflammatory conditions such as trauma and infection.

Interleukin-1 receptor antagonist (IL-1ra) is a 17 kDa member of the IL-1 family predominantly expressed in hepatocytes, epithelial cells, monocytes, macrophages, and neutrophils. IL-1ra has both intracellular and extracellular forms produced through alternative splicing. IL-1ra is thought to participate in the regulation of physiological IL-1 activity. The plasma concentration of IL-1ra is elevated in patients with acute myocardial infarction and unstable angina that proceeded to acute myocardial infarction, death, or refractory angina (Biasucci, L. M. et al., Circulation 99:2079-2084, 1999; Latini, R. et al., J. Cardiovasc. Pharmacol. 23:1-6, 1994). Furthermore, IL-1ra was significantly elevated in severe acute myocardial infarction as compared to uncomplicated acute myocardial infarction (Latini, R. et al., J. Cardiovasc. Pharmacol. 23:1-6, 1994).

Interleukin-6 (IL-6) is a 20 kDa secreted protein that is a hematopoietin family proinflammatory cytokine. Its major function is to mediate the acute phase production of hepatic proteins, and its synthesis is induced by the cytokine IL-1. IL-6 is normally produced by macrophages and T lymphocytes. The plasma concentration of IL-6 is elevated in patients with acute myocardial infarction and unstable angina, to a greater degree in acute myocardial infarction (Biasucci, L. M. et al., Circulation 94:874-877, 1996; Manten, A. et al., Cardiovasc. Res. 40:389-395, 1998; Biasucci, L. M. et al., Circulation 99:2079-2084, 1999). IL-6 is not significantly elevated in the plasma of patients with stable angina (Biasucci, L. M. et al., Circulation 94:874-877, 1996; Manten, A. et al., Cardiovasc. Res. 40:389-395, 1998). The plasma concentration of IL-6 is elevated within 8-12 hours of acute myocardial infarction onset, and can approach 100 pg/ml. The plasma concentration of IL-6 in patients with unstable angina was elevated at peak levels 72 hours after onset, possibly due to the severity of insult (Biasucci, L. M. et al., Circulation 94:874-877, 1996).

Interleukin-8 (IL-8) is a 6.5 kDa chemokine produced by monocytes, endothelial cells, alveolar macrophages and fibroblasts. IL-8 induces chemotaxis and activation of neutrophils and T cells.

Tumor necrosis factor α (TNFα) is a 17 kDa secreted proinflammatory cytokine that is involved in the acute phase response and is a pathogenic mediator of many diseases. TNF-alpha is a protein of 185 amino acids glycosylated at positions 73 and 172. It is synthesized as a precursor protein of 212 amino acids. Monocytes express at least five different molecular forms of TNF-alpha with molecular masses of 21.5-28 kDa. They mainly differ by post-translational alterations such as glycosylation and phosphorylation. The normal serum concentration of TNFα is <40 pg/ml (2 pM). The plasma concentration of TNFα is elevated in patients with acute myocardial infarction, and is marginally elevated in patients with unstable angina (Li, D. et al., Am. Heart J. 137:1145-1152, 1999; Squadrito, F. et al., Inflamm. Res. 45:14-19, 1996; Latini, R. et al., J. Cardiovasc. Pharmacol. 23:1-6, 1994; Carlstedt, F. et al., J. Intern. Med. 242:361-365, 1997). The concentration of TNFα in the plasma of acute myocardial infarction patients exceeded 300 pg/ml (15 pM) (Squadrito, F. et al., Inflamm. Res. 45:14-19, 1996).

Soluble intercellular adhesion molecule (sICAM-1), also called CD54, is a 85-110 kDa cell surface-bound immunoglobulin-like integrin ligand that facilitates binding of leukocytes to antigen-presenting cells and endothelial cells during leukocyte recruitment and migration. The plasma concentration of sICAM-1 is significantly elevated in patients with acute myocardial infarction and unstable angina, but not stable angina (Pellegatta, F. et al., J. Cardiovasc. Pharmacol. 30:455-460, 1997; Miwa, K. et al., Cardiovasc. Res. 36:37-44, 1997; Ghaisas, N. K. et al., Am. J. Cardiol. 80:617-619, 1997; Ogawa, H. et al., Am. J. Cardiol. 83:38-42, 1999). Furthermore, ICAM-1 is expressed in atherosclerotic lesions and in areas predisposed to lesion formation, so it may be released into the bloodstream upon plaque rupture (Iiyama, K. et al., Circ. Res. 85:199-207, 1999; Tenaglia, A. N. et al., Am. J. Cardiol. 79:742-747, 1997). Additional ICAM molecules are well known in the art, including ICAM-2 (also called CD102) and ICAM-3 (also called CD50), which may also be present in the blood.

Vascular cell adhesion molecule (VCAM), also called CD106, is a 100-110 kDa cell surface-bound immunoglobulin-like integrin ligand that facilitates binding of B lymphocytes and developing T lymphocytes to antigen-presenting cells during lymphocyte recruitment. The plasma concentration of sVCAM-1 is marginally elevated in patients with acute myocardial infarction, unstable angina, and stable angina (Mulvihill, N. et al., Am. J. Cardiol. 83:1265-7, A9, 1999; Ghaisas, N. K. et al., Am. J. Cardiol. 80:617-619, 1997). However, sVCAM-1 is expressed in atherosclerotic lesions and its plasma concentration may correlate with the extent of atherosclerosis (Iiyama, K. et al., Circ. Res. 85:199-207, 1999; Peter, K. et al., Arterioscler. Thromb. Vasc. Biol. 17:505-512, 1997).

Macrophage migration inhibitory factor (MIF) is a lymphokine involved in cell-mediated immunity, immunoregulation, and inflammation. Like TNFα and IL-1β, MIF plays a central role in the host response to endotoxemia. Coinjection of recombinant MIF and LPS exacerbates LPS lethality, whereas neutralizing anti-MIF antibodies fully protect mice from endotoxic shock.

Hemoglobin (Hb) is an oxygen-carrying iron-containing globular protein found in erythrocytes. It is a heterodimer of two globin subunits. α₂γ₂ is referred to as fetal Hb, α₂β₂ is called adult HbA, and α₂δ₂ is called adult HbA₂. 90-95% of hemoglobin is HbA, and the α₂ globin chain is found in all Hb types, even sickle cell hemoglobin. Hb is responsible for carrying oxygen to cells throughout the body. Hbα₂ is not normally detected in serum.

Oxysterols (oxidized derivatives of cholesterol) and oxidized lipoproteins have been identified in atherosclerotic lesions, and are suggested to play a role in the pathogenesis of coronary heart disease. See, e.g., Staprans et al., Arterioscler. Thromb. Vasc. Biol. 20: 708-14, 2000. Recently, an aldol condensation product believed to be formed by ozonolysis of cholesterol in atherosclerotic plaques was reported to be detectable in plasma from subjects with advanced atherosclerotic disease. It was suggested that this molecule may be a marker of arterial inflammation in atherosclerosis. Wentworth et al., Science 302: 1053-6, 2003. This publication is hereby incorporated by reference in its entirety.

Human lipocalin-type prostaglandin D synthase (hPDGS), also called β-trace, is a 30 kDa glycoprotein that catalyzes the formation of prostaglandin D2 from prostaglandin H. Elevations of hPDGS have been identified in blood from patients with unstable angina and cerebral infarction (Patent No. EP0999447A1). Furthermore, hPDGS appears to be a useful marker of ischemic episodes (Patent No. EP0999447A1).

Mast cell tryptase, also known as alpha tryptase, is a 275 amino acid (30.7 kDa) protein that is the major neutral protease present in mast cells. Mast cell tryptase is a specific marker for mast cell activation, and is a marker of allergic airway inflammation in asthma and in allergic reactions to a diverse set of allergens. See, e.g., Taira et al., J. Asthma 39: 315-22 (2002); Schwartz et al., N. Engl. J. Med. 316: 1622-26 (1987). Elevated serum tryptase levels (>1 ng/mL) between 1 and 6 hours after an event provides a specific indication of mast cell degranulation.

Eosinophil cationic protein (ECP) is a heterogeneous protein with molecular weight variants from 16-24 kDa and a pI of pH 10.8. Assessment of serum ECP may be assumed to reflect pulmonary inflammation in bronchial asthma. Koller et al., Arch. Dis. Childhood 73: 413-7 (1995); see also, Sorkness et al., Clin. Exp. Allergy 32: 1355-59 (2002); Badr-elDin et al., East Mediterr. Health J. 5: 664-75 (1999).

KL-6 (also referred to as MUC1) is a high molecular weight (>300 kDa) mucinous glycoprotein expressed on pneumonocytes. Serum levels of KL-6 are reportedly elevated in interstitial lung diseases, which are characterized by exertional dyspnea. KL-6 has been shown to be a marker of various interstitial lung diseases, including pulmonary fibrosis, interstitial pneumonia, sarcoidosis, and interstitial pneumonitis. See, e.g., Kobayashi and Kitamura, Chest 108: 311-15 (1995); Kohno, J. Med. Invest. 46: 151-58 (1999); Bandoh et al., Ann. Rheum. Dis. 59: 257-62 (2000); and Yamane et al., J. Rheumatol. 27: 930-4 (2000).

Interleukin 10 (“IL-10”) is a 160 amino acid (18.5 kDa predicted mass) cytokine that is a member of the four α-helix bundle family of cytokines. In solution, IL-10 forms a homodimer having an apparent molecular weight of 39 kDa. The human IL-10 gene is located on chromosome 1. Viera et al., Proc. Natl. Acad. Sci. USA 88: 1172-76 (1991); Kim et al., J. Immunol. 148: 3618-23 (1992). Overproduction of IL-10 has been identified as a marker in sepsis, and is predictive of severity and mortality. Gogos et al., J. Infect. Dis. 181: 176-80 (2000).

(v) Exemplary Specific Markers of Neural Tissue Injury

In the case where the vascular disease affects tissues other than myocardium (e.g., in stroke), specific markers of tissue damage other than markers of myocardial tissue damage may be particularly useful. Considering stroke as an example, the following list of exemplary specific markers of neural tissue injury is provided. This list is not meant to be limiting.

Adenylate kinase (AK) is a ubiquitous 22 kDa cytosolic enzyme that catalyzes the interconversion of ATP and AMP to ADP. Four isoforms of adenylate kinase have been identified in mammalian tissues (Yoneda, T. et al., Brain Res Mol Brain Res 62:187-195, 1998). The AKI isoform is found in brain, skeletal muscle, heart, and aorta. Serum AKI appears to have the greatest specificity of the AK isoforms as a marker of neural tissue injury. AK may be best suited as a cerebrospinal fluid marker of cerebral ischemia, where its dominant source would be neural tissue.

Neurotrophins are a family of growth factors expressed in the mammalian nervous system. Some examples include nerve growth factor (NGF), brain-derived neurotrophic factor (BDNF), neurotrophin-3 (NT-3) and neurotrophin-4/5 (NT-4/5). Neurotrophins exert their effects primarily as target-derived paracrine or autocrine neurotrophic factors. The role of the neurotrophins in survival, differentiation and maintenance of neurons is well known. They exhibit partially overlapping but distinct patterns of expression and cellular targets. In addition to the effects in the central nervous system, neurotrophins also affect peripheral afferent and efferent neurons.

BDNF is a potent neurotrophic factor which supports the growth and survivability of nerve and/or glial cells. BDNF is expressed as a 32 kDa precursor “pro-BDNF” molecule that is cleaved to a mature BDNF form. Mowla et al., J. Biol. Chem. 276: 12660-6 (2001). The most abundant active form of human BDNF is a 27 kDa homodimer, formed by two identical 119 amino acid subunits, which is held together by strong hydrophobic interactions; however, pro-BDNF is also released extracellularly and is biologically active.

NT-3 is also a 27 kDa homodimer consisting of two 119-amino acid subunits. The addition of NT-3 to primary cortical cell cultures has been shown to exacerbate neuronal death caused by oxygen-glucose deprivation, possible via oxygen free radical mechanisms (Bates et al., Neurobiol. Dis. 9:24-37, 2002). NT-3 is expressed as an inactive pro-NT-3 molecule, which is cleaved to the mature biologically active form.

Calbindin-D is a 28 kDa cytosolic vitamin D-dependent Ca²⁺-binding protein that may serve a cellular protective function by stabilizing intracellular calcium levels. Calbindin-D is found in the central nervous system, mainly in glial cells, and in cells of the distal renal tubule (Hasegawa, S. et al., J. Urol. 149:1414-1418, 1993). The normal serum concentration of calbindin-D is <20 pg/ml (0.7 pM). Serum calbindin-D concentration is reportedly elevated following cardiac arrest, and this elevation is thought to be a result of CNS damage due to cerebral ischemia (Usui, A. et al., J. Neurol. Sci. 123:134-139, 1994). Elevations of serum calbindin-D are elevated and plateau soon after reperfusion following ischemia. Maximum serum calbindin-D concentrations can be as much as 700 pg/ml (25 pM).

Creatine kinase (CK) is a cytosolic enzyme that catalyzes the reversible formation of ADP and phosphocreatine from ATP and creatine. The brain-specific CK isoform (CK-BB) is an 85 kDa cytosolic protein that accounts for approximately 95% of the total brain CK activity. It is also present in significant quantities in cardiac tissue, intestine, prostate, rectum, stomach, smooth muscle, thyroid uterus, urinary bladder, and veins (Johnsson, P. J., Cardiothorac. Vasc. Anesth. 10:120-126, 1996). Elevations of CK-BB in serum can be attributed to neural tissue injury due to ischemia, coupled with increased permeability of the blood brain barrier. In severe stroke, serum concentrations CK-BB are elevated and peak soon after the onset of stroke (within 24 hours), gradually returning to normal after 3-7 days (4).

Glial fibrillary acidic protein (GFAP) is a 55 kDa cytosolic protein that is a major structural component of astroglial filaments and is the major intermediate filament protein in astrocytes. GFAP is specific to astrocytes, which are interstitial cells located in the CNS and can be found near the blood-brain barrier. Serum GFAP is elevated following ischemic stroke (Niebroj-Dobosz, I., et al., Folia Neuropathol. 32:129-137, 1994). Serum concentrations GFAP appear to be elevated soon after the onset of stroke, continuously increase and persist for an amount of time (weeks) that may correlate with the severity of damage.

Lactate dehydrogenase (LDH) is a ubiquitous 135 kDa cytosolic enzyme. It is a tetramer of A and B chains that catalyzes the reduction of pyruvate by NADH to lactate. Five isoforms of LDH have been identified in mammalian tissues, and the tissue-specific isoforms are made of different combinations of A and B chains. Elevations in serum LDH activity are reported following both ischemic and hemorrhagic stroke, but further studies are needed in serum to confirm this observation and to determine a correlation with the severity of injury and neurological outcome (Aggarwal, S. P. et al., J. Indian Med. Assoc. 93:331-332, 1995; Maiuri, F. et al., Neurol. Res. 11:6-8, 1989).

Myelin basic protein (MBP) is actually a 14-21 kDa family of cytosolic proteins generated by alternative splicing of a single MBP gene that is likely involved in myelin compaction around axons during the myelination process. MBP is specific to oligodendrocytes in the CNS and in Schwann cells of the peripheral nervous system (PNS). Serum MBP is elevated after all types of severe stroke, specifically thrombotic stroke, embolic stroke, intracerebral hemorrhage, and subarachnoid hemorrhage, while elevations in MBP concentration are not reported in the serum of individuals with strokes of minor to moderate severity, which would include lacunar infarcts or transient ischemic attacks (Palfreyman, J. W. et al., Clin. Chim. Acta 92:403-409, 1979). The serum concentration of MBP has been reported to correlate with the extent of damage (infarct volume), and it may also correlate with neurological outcome.

Neural cell adhesion molecule (NCAM), also called CD56, is a 170 kDa cell surface-bound immunoglobulin-like integrin ligand that is involved in the maintenance of neuronal and glial cell interactions in the nervous system, where it is expressed on the surface of astrocytes, oligodendrocytes, Schwann cells, neurons, and axons. NCAM is also localized to developing skeletal muscle myotubes, and its expression is upregulated in skeletal muscle during development, denervation and renervation.

Proteolipid protein (PLP) is a 30 kDa integral membrane protein that is a major structural component of CNS myelin. PLP is specific to oligodendrocytes in the CNS and accounts for approximately 50% of the total CNS myelin protein in the central sheath, although extremely low levels of PLP have been found (<1%) in peripheral nervous system (PNS) myelin. Serum PLP is elevated after cerebral infarction, but not after transient ischemic attack (Trotter, J. L. et al., Ann. Neurol. 14:554-558, 1983). Elevations of PLP in serum can be attributed to neural tissue injury due to physical damage or ischemia caused by infarction or cerebral hemorrhage, coupled with increased permeability of the blood brain barrier.

S-100β is elevated in serum after 4 hours from stroke onset, with concentrations reaching a maximum 2-3 days after onset. After the serum concentration reaches its maximum, which can approach 20 ng/ml (1.9 mM), it gradually decreases to normal over approximately one week. Because the severity of damage has a direct effect on the release of S-100β, it will affect the release kinetics by influencing the length of time that S-100β is elevated in the serum. S-100β will be present in the serum for a longer period of time as the seventy of injury increases. Furthermore, elevated serum concentrations of S-100β can indicate complications related to neural tissue injury after AMI and cardiac surgery.

Thrombomodulin (TM) is a 70 kDa single chain integral membrane glycoprotein found on the surface of vascular endothelial cells. Current reports describing serum TM concentration alterations following ischemic stroke are mixed, reporting no changes or significant increases (Seki, Y. et al., Blood Coagul. Fibrinolysis 8:391-396, 1997). Serum elevations of TM concentration reflect endothelial cell injury and would not indicate coagulation or fibrinolysis activation.

The gamma isoform of protein kinase C (PKCg) is specific for CNS tissue and is not normally found in the circulation. PKCg is activated during cerebral ischemia and is present in the ischemic penumbra at levels 2-24-fold higher than in contralateral tissue, but is not elevated in infarcted tissue (Krupinski, J. et al., Acta Neurobiol. Exp. (Warz) 58:13-21, 1998). Additional isoforms of PKC, beta I and beta II were found in increased levels in the infarcted core of brain tissue from patients with cerebral ischemia (Krupinski, J. et al., Acta Neurobiol. Exp. (Warz) 58:13-21, 1998). Furthermore, the alpha and delta isoforms of PKC (PKCa and PKCd, respectively) have been implicated in the development of vasospasm following subarachnoid hemorrhage using a canine model of hemorrhage. Therefore, it may be of benefit to measure various isoforms of PKC, either individually or in various combinations thereof, for the identification of cerebral damage, the presence of the ischemic penumbra, as well as the development and progression of cerebral vasospasm following subarachnoid hemorrhage. Ratios of PKC isoforms such as PKCg and either PKCbI, PKCbII, or both also may be of benefit in identifying a progressing stroke, where the ischemic penumbra is converted to irreversibly damaged infarcted tissue.

(vi) Non-Specific Markers for Cellular Injury

Human vascular endothelial growth factor (VEGF) is a dimeric protein, the reported activities of which include stimulation of endothelial cell growth, angiogenesis, and capillary permeability. VEGF is secreted by a variety of vascularized tissues. In an oxygen-deficient environment, vascular endothelial cells may be damaged and may not ultimately survive. However, such endothelial damage stimulates VEGF production by vascular smooth muscle cells. Vascular endothelial cells may exhibit increased survival in the presence of VEGF, an effect that is believed to be mediated by expression of Bcl-2. VEGF can exist as a variety of splice variants known as VEGF(189), VEGF(165), VEGF(164), VEGFB(155), VEGF(148), VEGF(145), and VEGF(121).

Insulin-like growth factor-1 (IGF-1) is a ubiquitous 7.5 kDa secreted protein that mediates the anabolic and somatogenic effects of growth hormone during development (1, 2). In the circulation, IGF-1 is normally bound to an IGF-binding protein that regulates IGF activity. Serum IGF-1 concentrations are reported to be significantly decreased in individuals with ischemic stroke, and the magnitude of reduction appears to correlate with the severity of injury (Schwab, S. et al., Stroke 28:1744-1748, 1997). Serum IGF-1 may be a sensitive indicator of neural tissue injury. However, the ubiquitous expression pattern of IGF-1 indicates that all tissues can potentially affect serum concentrations of IGF-1.

Adhesion molecules are involved in the inflammatory response can also be considered as acute phase reactants, as their expression levels are altered as a result of insult. Examples of such adhesion molecules include E-selectin, intercellular adhesion molecule-1, vascular cell adhesion molecule, and the like.

E-selectin, also called ELAM-1 and CD62E, is a 140 kDa cell surface C-type lectin expressed on endothelial cells in response to IL-1 and TNFα that mediates the “rolling” interaction of neutrophils with endothelial cells during neutrophil recruitment. Some investigations report increases in serum E-selectin concentration following ischemic stroke, while others find it unchanged (Bitsch, A. et al., Stroke 29:2129-2135, 1998; Kim, J. S., J. Neurol. Sci. 137:69-78, 1996; Shyu, K. G. et al., J. Neurol. 244:90-93, 1997). E-selectin concentrations are elevated in the CSF of individuals with subarachnoid hemorrhage and may predict vasospasm (Polin, R. S. et al., J. Neurosurg. 89:559-567, 1998). Serum E-selectin concentrations are elevated in individuals with atherosclerosis, various forms of cancer, preeclampsia, diabetes, cystic fibrosis, AMI, and other nonspecific inflammatory states (Hwang, S. J. et al., Circulation 96:4219-4225, 1997; Banks, R. E. et al., Br. J. Cancer 68:122-124, 1993; Austgulen, R. et al., Eur. J. Obstet. Gynecol. Reprod. Biol. 71:53-58, 1997; Steiner, M. et al., Thromb. Haemost. 72:979-984, 1994; De Rose, V. et al., Am. J. Respir. Crit. CareMed. 157:1234-1239, 1998).

Head activator (HA) is an 11 amino acid, 1.1 kDa neuropeptide that is found in the hypothalamus and intestine. It was originally found in the freshwater coelenterate hydra, where it acts as a head-specific growth and differentiation factor.

Glycated hemoglobin HbA1c measurement provides an assessment of the degree to which blood glucose has been elevated over an extended time period, and so has been related to the extent diabetes is controlled in a patient. Glucose binds slowly to hemoglobin A, forming the A1c subtype. The reverse reaction, or decomposition, proceeds relatively slowly, so any buildup persists for roughly 4 weeks. With normal blood glucose levels, glycated hemoglobin is expected to be 4.5% to 6.7%. As blood glucose concentration rise, however, more binding occurs. Poor blood sugar control over time is suggested when the glycated hemoglobin measure exceeds 8.0%.

(vii) Markers Related to Apoptosis

Apoptosis refers to the eukaryotic “programmed cell death” pathway. The pathway is dependent upon intracellular proteases and nucleases, leading ultimately to fragmentation of genomic DNA and cell death. The following exemplary list of markers related to apoptosis is not meant to be limiting.

Caspases are a family of proteases that relay a “doomsday” signal in a step-wise manner reminiscent of signaling by kinases. Caspases are present in all cells as latent enzymes. They are recruited to receptor-associated cytosolic complexes whose formation is initiated by receptor oligomerization (e.g., TNF receptors, FAS, and TRAIL receptors) and to other cytoplasmic adaptor proteins, such as APAF-1. Recruitment of caspases to oligomerized receptors leads to activation via dimerization or dimerization accompanied by autoproteolytic cleavage. Active caspases can proteolyze additional caspases generating a caspase cascade that cleaves proteins critical for cell survival. The final outcome of this signaling pathway is a form of controlled cell death termed apoptosis.

The subgroup of caspases involved in apoptosis has been referred to as either initiators or effectors. Caspases-8, -9, and -10 (possibly, -2 and -5) can initiate the caspase activation cascade and are therefore called initiators. Based on the prototypes, caspases-8 and -9, initiators can be activated either by dimerization alone (caspase-9) or by dimerization with concomitant autoproteolysis (caspase-8). The effector caspases-3, -6, and -7 propagate the cascade and are activated by proteolytic cleavage by other caspases. Although an initiator caspase may not be responsible for starting the caspase cascade, it can become activated and involved in later steps of the cascade. Thus, in the latter scenario, the caspase would be more appropriately termed an effector.

Caspase-3, also called CPP-32, YAMA, and apopain, is an interleukin-1β converting enzyme (ICE)-like intracellular cysteine proteinase that is activated during cellular apoptosis. Caspase-3 is present as an inactive 32 kDa precursor that is proteolytically activated during apoptosis induction into a heterodimer of 20 kDa and 11 kDa subunits (Femandes-Alnemri, T. et al., J. Biol. Chem. 269:30761-30764, 1994). Its cellular substrates include poly(ADP-ribose) polymerase (PARP) and sterol regulatory element binding proteins (SREBPS) (Liu, X. et al., J. Biol. Chem. 271:13371-13376, 1996). The normal plasma concentration of caspase-3 is unknown. There are no published investigations into changes in the plasma concentration of caspase-3 associated with ACS. There are increasing amounts of evidence supporting the hypothesis of apoptosis induction in cardiac myocytes associated with ischemia and hypoxia (Saraste, A., Herz 24:189-195, 1999; Ohtsuka, T. et al., Coron. Artery Dis. 10:221-225, 1999; James, T. N., Coron. Artery Dis. 9:291-307, 1998; Bialik, S. et al., J. Clin. Invest. 100:1363-1372, 1997; Long, X. et al., J. Clin. Invest. 99:2635-2643, 1997). Elevations in the plasma caspase-3 concentration may be associated with any physiological event that involves apoptosis. There is evidence that suggests apoptosis is induced in skeletal muscle during and following exercise and in cerebral ischemia (Carraro, U. and Franceschi, C., Aging (Milano) 9:19-34, 1997; MacManus, J. P. et al., J. Cereb. Blood Flow Metab. 19:502-510, 1999).

Cathepsin D (E.C.3.4.23.5.) is a soluble lysosomal aspartic proteinase. It is synthesized in the endoplasmic reticulum as a preprocathepsin D. Having a mannose-6-phosphate tag, procathepsin D is recognized by a mannose-6-phosphate receptor. Upon entering into an acidic lysosome, the single-chain procathepsin D (52 KDa) is activated to cathepsin D and subsequently to a mature two-chain cathepsin D (31 and 14 KDa, respectively). The two mannose-6-phosphate receptors involved in the lysosomal targeting of procathepsin D are expressed both intracellularly and on the outer cell membrane. The glycosylation is believed to be crucial for normal intracellular trafficking. The fundamental role of cathepsin D is to degrade intracellular and internalized proteins. Cathepsin D has been suggested to take part in antigen processing and in enzymatic generation of peptide hormones. The tissue-specific function of cathepsin D seems to be connected to the processing of prolactin. Rat mammary glands use this enzyme for the formation of biologically active fragments of prolactin. Cathepsin D is functional in a wide variety of tissues during their remodeling or regression, and in apoptosis.

Brain α spectrin (also referred to as α fodrin) is a cytoskeletal protein of about 284 kDa that interacts with calmodulin in a calcium-dependent manner. Like erythroid spectrin, brain α spectrin forms oligomers (in particular dimers and tetramers). Brain α spectrin contains two EF-hand domains and 23 spectrin repeats. The caspase 3-mediated cleavage of α spectrin during apoptotic cell death may play an important role in altering membrane stability and the formation of apoptotic bodies.

Other Preferred Markers

The following table provides a list of additional preferred markers, associated with a disease or condition for which each marker can provide useful information for differential diagnosis. Various markers may be listed for more than one condition. As understood by the skilled artisan and described herein, markers may indicate different conditions when considered with additional markers in a panel; alternatively, markers may indicate different conditions when considered in the entire clinical context of the patient.

| Marker | Classification |
|---|---|
| Haptoglobin | Inflammatory |
| Hepcidin | Acute phase reactant |
| HSP-60 | Acute phase reactant |
| HSP-65 | Acute phase reactant |
| HSP-70 | Acute phase reactant |
| Myoglobin | Myocardial injury |
| PAPPA | Inflammatory |
| PECAM 1 | Acute phase reactant |
| Prostaglandin-D-Synthetase | Marker of ischemia |
| S100□ | Myocardial injury |
| s-CD40 ligand* | Inflammatory |
| S-FAS ligand | Acute phase reactant |
| Troponin I and complexes | Myocardial injury |
| cardiotrophin 1 | Inflammatory |
| urotensin II | Blood pressure regulation |
| asymmetric dimethylarginine | Acute phase reactant |
| BNP | Blood pressure regulation |
| Fibrinogen | coagulation and hemostasis |
| ANP | Blood pressure regulation |
| CNP | Blood pressure regulation |
| Ubiquitin Fusion Degradation | |
| Protein I Homolog | Apoptosis |
| alpha 2 actin | Vascular tissue |
| basic calponin 1 | Vascular tissue |
| beta like 1 integrin | Vascular tissue |
| Calponin | Vascular tissue |
| CSRP2 | Vascular tissue |
| elastin | Vascular tissue |
| Fibrillin 1 | Vascular tissue |
| LTBP4 | Vascular tissue |
| smooth muscle myosin | Vascular tissue |
| transgelin | Vascular tissue |
| calcitonin gene related peptide | Blood pressure regulation |
| Carboxyterminal propeptide of | Marker of collagen synthesis |
| type I procollagen (PICP) | |
| Collagen carboxyterminal | Marker of collagen degradation |
| telopeptide (ICTP) | |
| Fibronectin | Inflammatory |
| MMP-11 | Acute phase reactant |
| MMP-3 | Acute phase reactant |
| MMP-9 | Acute phase reactant |
| arg-Vasopressin | Blood pressure regulation |
| aldosterone | Blood pressure regulation |
| angiotensin 1 | Blood pressure regulation |
| angiotensin 2 | Blood pressure regulation |
| angiotensin 3 | Blood pressure regulation |
| Antithrombin-III | coagulation and hemostasis |
| Bradykinin | Blood pressure regulation |
| calcitonin | Blood pressure regulation |
| Endothelin-2 | Blood pressure regulation |
| Endothelin-3 | Blood pressure regulation |
| Renin | Blood pressure regulation |
| Urodilatin | Blood pressure regulation |
| Defensin HBD 1 | Acute phase reactant |
| Defensin HBD 2 | Acute phase reactant |
| alpha enolase | Pulmonary tissue specific |
| LAMP 3 | Pulmonary tissue specific |
| LAMP3 | Pulmonary tissue specific |
| Lung Surfactant protein D | Pulmonary tissue specific |
| phospholipase D | Pulmonary tissue specific |
| PLA2G5 | Pulmonary tissue specific |
| SFTPC | Pulmonary tissue specific |
| D-dimer | coagulation and hemostasis |
| HMG | Inflammatory |
| IL-1 | Inflammatory |
| IL-8 | Inflammatory |
| IL-10* | Inflammatory |
| IL-11* | Inflammatory |
| IL-13* | Inflammatory |
| IL-18* | Inflammatory |
| IL-4* | Inflammatory |
| macrophage inhibitory factor | Inflammatory |
| s-acetyl Glutathione | apoptosis |
| Serum Amyloid A | Acute phase reactant |
| s-iL 18 receptor | pro and anti-Inflammatory modulator |
| S-iL-1 receptor | pro and anti-Inflammatory modulator |
| s-TNF P55 | Inflammatory and growth factor |
| s-TNF P75 | Inflammatory and growth factor |
| TGF-beta | Acute phase reactant |
| MMP-11 | Acute phase reactant |
| PAI-1 | coagulation and hemostasis |
| Procalcitonin | Blood pressure regulation |
| PROTEIN C | coagulation and hemostasis |
| TAFI | coagulation and hemostasis |
| CRP | Acute phase reactant |
| e-selectin | Acute phase reactant |
| 14-3-3 | Neural tissue injury |
| 4.1B | Neural tissue injury |
| adrenomedullin | Blood pressure regulation |
| APO E4-1 | Neural tissue injury |
| Atrophin 1 | Neural tissue injury |
| Beta NGF | Acute phase reactant |
| beta thromboglobulin | coagulation and hemostasis |
| BNP | Blood pressure regulation |
| brain Derived neurotrophic | Neural tissue injury |
| factor | |
| Brain Fatty acid binding protein | Neural tissue injury |
| brain tubulin | Neural tissue injury |
| CACNA1A | Neural tissue injury |
| Calbindin D | Neural tissue injury |
| Calbrain | Neural tissue injury |
| calcyphosine | Blood pressure regulation |
| Carbonic anhydrase XI | Neural tissue injury |
| Caspase 3 | apoptosis |
| Cathepsin D | apoptosis |
| CBLN1 | Neural tissue injury |
| CD44 | Inflammatory |
| Cerebellin 1 | Neural tissue injury |
| Chimerin 1 | Neural tissue injury |
| Chimerin 2 | Neural tissue injury |
| CHN 1 | Neural tissue injury |
| CHN 2 | Neural tissue injury |
| Ciliary neurotrophic factor | Neural tissue injury |
| CKBB | Neural tissue injury |
| CNP | Blood pressure regulation |
| CRHR1 | Neural tissue injury |
| C-tau | Neural tissue injury |
| cytochrome C | apoptosis |
| DRPLA | Neural tissue injury |
| EGF | Inflammatory and growth factors |
| Endothelin-1 | Blood pressure regulation |
| E-selectin | Acute phase reactant |
| Fibrinopeptide A | coagulation and hemostasis |
| Fibronectin | Inflammatory |
| GFAP | Neural tissue injury |
| Glutathione S Transferase | Acute phase reactant |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIF 1 ALPHA | Acute phase reactant |
| HIP2 | Neural tissue injury |
| HSP-60 | Acute phase reactant |
| IL-10 | Inflammatory |
| IL-1-Beta | Inflammatory |
| IL-1ra | Inflammatory |
| IL-6 | Inflammatory |
| IL-8 | Inflammatory |
| I-NOS | Acute phase reactant |
| Insulin-like growth factor | Inflammatory |
| Intracellular adhesion molecule | Acute phase reactant |
| KCNK4 | Neural tissue injury |
| KCNK9 | Neural tissue injury |
| KCNQ5 | Neural tissue injury |
| Lactate dehydrogenase | Acute phase reactant |
| MAPK10 | Neural tissue injury |
| MCP-1 | Acute phase reactant |
| MDA-LDL | plaque rupture |
| MMP-3 | Acute phase reactant |
| MMP-9 | Acute phase reactant |
| myelin basic protein | Neural tissue injury |
| n-acetyl aspartate | Acute phase reactant |
| NCAM | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1,2,3,4 | Neural tissue injury |
| NRG2 | Neural tissue injury |
| Osteoprotegerin | Inflammatory |
| PACE4 | Neural tissue injury |
| phosphoglycerate mutase | Neural tissue injury |
| PKC gamma | Neural tissue injury |
| Plasmin alpha 2 antiplasmin | |
| complex | coagulation and hemostasis |
| Platelet factor 4 | coagulation and hemostasis |
| Prostaglandin D-synthase | Acute phase reactant |
| Prostaglandin E2 | Acute phase reactant |
| proteolipid protein | Neural tissue injury |
| PTEN | Neural tissue injury |
| PTPRZ1 | Neural tissue injury |
| RANK ligand | Acute phase reactant |
| RGS9 | Neural tissue injury |
| RNA Binding protein Regulatory | |
| Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| spectrin | apoptosis |
| SREB3 | Neural tissue injury |
| STAC | Neural tissue injury |
| STX1A | Neural tissue injury |
| STXBP1 | Neural tissue injury |
| Syntaxin | Neural tissue injury |
| Thrombin antithrombin III | coagulation and hemostasis |
| complex | |
| Thrombomodulin | coagulation and hemostasis |
| Thrombus Precursor Protein | coagulation and hemostasis |
| Tissue factor | coagulation and hemostasis |
| TNF Receptor Superfamily | Acute phase reactant |
| Member 1A | |
| Transforming growth factor beta | Inflammatory |
| transthyretin | Neural tissue injury |
| Tumor necrosis factor alpha | Acute phase reactant |
| Vascular cell adhesion molecule | Acute phase reactant |
| Vascular endothelial growth | Inflammatory |
| factor | |
| von Willebrand factor | coagulation and hemostasis |
| adenylate kinase-1 | Neural tissue injury |
| BDNF* | Neural tissue injury |
| CGRP | Blood pressure regulation |
| cystatin C | Acute phase reactant |
| neurokinin A | Neural tissue injury |
| substance P | Inflammatory |
| D Dimer | coagulation and hemostasis |
| Myeloperoxidase (MPO) | Inflammatory |
| Oxidized Low-Density | |
| Lipoproteins (OxLDL) | markers of atherosclerosis |

Ubiguitination of Markers

Ubiquitin-mediated degradation of proteins plays an important role in the control of numerous processes, such as the way in which extracellular materials are incorporated into a cell, the movement of biochemical signals from the cell membrane, and the regulation of cellular functions such as transcriptional on-off switches. The ubiquitin system has been implicated in the immune response and development. Ubiquitin is a 76-amino acid polypeptide that is conjugated to proteins targeted for degradation. The ubiquitin-protein conjugate is recognized by a 26S proteolytic complex that splits ubiquitin from the protein, which is subsequently degraded. Levels of ubiquitinated proteins generally, or of specific ubiquitin-protein conjugates or fragments thereof, can be measured as additional markers of the invention. Moreover, circulating levels of ubiquitin itself can be a useful marker in the methods described herein. See, e.g., Hu et al., J. Cereb. Blood Flow Metab. 21: 865-75, 2001.

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of a marker of interest could use one antibody (on a solid phase or label) that recognizes ubiquitin, and a second antibody (the other of the solid phase or label) that recognizes the marker protein.

The present invention contemplates measuring ubiquitin conjugates of any marker described herein.

Identification of Marker Panels

Methods and systems for the identification of one or more markers for the diagnosis, and in particular for the differential diagnosis, of disease have been described previously. Suitable methods for identifying markers useful for the diagnosis of disease states are described in detail in U.S. Provisional Patent Application No. 60/436,392, entitled METHOD AND SYSTEM FOR DISEASE DETECTION USING MARKER COMBINATIONS (attorney docket no. 071949-6801), filed Dec. 24, 2002, and U.S. patent application Ser. No. 10/331,127, entitled METHOD AND SYSTEM FOR DISEASE DETECTION USING MARKER COMBINATIONS (attorney docket no. 071949-6802), filed Dec. 27, 2002, each of which is hereby incorporated by reference in its entirety, including all tables, figures, and claims. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those ACS patients that have recently had a subsequent adverse outcome. Hereinafter, subjects in this first set will be referred to as “diseased”.

The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects. In the case of the ACS example described hereinafter, the “non-diseased” group may be those ACS patients that, over the same time period, did not suffer a subsequent adverse outcome.

The data obtained from subjects in these sets includes levels of a plurality of markers, including TpP™ and/or MCP-1. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for a each subject 0) is expressed as:

Rⱼ=ΣwᵢI_{i,j},

where i is the marker index, j is the subject index, wᵢ is the weighting coefficient for marker i, I is the indicator value to which the marker level for marker i is mapped for subject j, and Σ is the summation over all candidate markers i.

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels that require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

One cound use such a method to define new “markers” based on panel responses, and even to determine a “panel response of panel responses.” For example, one may divide ACS and non-ACS subjects as follows: (1) ACS+adverse outcome; (2) ACS−adverse outcome; (3) normals. Obne would define a first panel constructed from a plurality of markers as described above, and obtain the panel responses from this first panel for all the subjects. Then, the members of any one of these 3 groups may be compared to the panel responses of the members of any other of these groups to define a function and weighting factor that best differentiates these two groups based on the panel responses. This can be repeated as all 3 groups are compared pairwise. The “markers” used to define a second panel might include any or all of the following as a new “marker”: (1) versus (2) as marker 1; (1) versus (3) as marker 2; (2) versus (3) as marker 3.

Measures of test accuracy may be obtained as described in Fischer et al., Intensive Care Med. 29: 1043-51, 2003; Zhou et al., Statistical Methods in Diagnostic Medicine, John Wiley & Sons, 2002; and Motulsky, Intuitive Biostatistics, Oxford University Press, 1995; and other publications well known to those of skill in the art, and used to determine the effectiveness of a given marker or panel of markers. These measures include sensitivity and specificity, predictive values, likelihood ratios, diagnostic odds ratios, hazard ratios, and ROC curve areas. As discussed above, suitable tests may exhibit one or more of the following results on these various measures:

A ROC curve area of greater than about 0.5, more preferably greater than about 0.7, still more preferably greater than about 0.8, even more preferably greater than about 0.85, and most preferably greater than about 0.9;

a positive or negative likelihood ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less;

an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; and/or

a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less.

Measures of diagnostic accuracy such as those discussed above are often reported together with confidence intervals or p values. These may be calculated by methods well known in the art. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. No. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of ACS, identification of the severity of the event, identification of the disease severity, and identification of the patient's outcome, including risk of future events.

A panel consisting of the markers referenced above may be constructed to provide relevant information related to differential diagnosis and/or prognosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W. B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting a Treatment Regimen

The appropriate treatments for various types of vascular disease may be large and diverse. However, once a diagnosis is obtained, the clinician can readily select a treatment regimen that is compatible with the diagnosis. Accordingly, the present invention provides methods of early differential diagnosis to allow for appropriate intervention in acute time windows. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, NJ, 1999.

### EXAMPLES

The following examples serve to illustrate the present invention. These examples are in no way intended to limit the scope of the invention.

### Example 1

### Blood Sampling

Blood specimens were collected by trained study personnel. Samples were collected and processed as described previously. See, de Lemos et al., The prognostic value of B-type natriuretic peptide in patients with acute coronary syndromes, N Engl J Med 345:1014-21 (2001). American Biogenetic Sciences, Inc. instructs users of the TpP™ ELISA assay kit to collect blood using citrate as an anticoagulant, and they recommend against using EDTA. Plasma samples were collected in citrate anticoagulant and frozen at the study site at −20° C. or colder within 60 minutes of collection. The specimens were shipped on dry ice to the TIMI Cardiac Marker Core Laboratory at Children's Hospital (Boston, Mass.) where they were stored at −70° C. Following completion of the OPUS-TIMI 16 trial, all plasma specimens from the 50/50 treatment arm were shipped on dry ice to BIOSITE, Inc. (San Diego, Calif.), where assays were performed.

### Example 2

### Biochemical Analyses

Markers were typically measured using standard immunoassay techniques. These techniques involved the use of antibodies to specifically bind the protein targets. A monoclonal antibody directed against a selected marker was biotinylated using N-hydroxysuccinimide biotin (NHS-biotin) at a ratio of about 5 NHS-biotin moieties per antibody. The antibody-biotin conjugate was then added to wells of a standard avidin 384 well microtiter plate, and antibody conjugate not bound to the plate was removed. This formed the “anti-marker” in the microtiter plate. Another monoclonal antibody directed against the same marker was conjugated to alkaline phosphatase using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Immunoassays were performed on a TECAN Genesis RSP 200/8 Workstation. Biotinylated antibodies were pipetted into microtiter plate wells previously coated with avidin and incubated for 60 min. The solution containing unbound antibody was removed, and the cells were washed with a wash buffer, consisting of 20 mM borate (pH 7.42) containing 150 mM NaCl, 0.1% sodium azide, and 0.02% Tween-20. The plasma samples (10 μL) were pipeted into the microtiter plate wells, and incubated for 60 min. The sample was then removed and the wells were washed with a wash buffer. The antibody-alkaline phosphatase conjugate was then added to the wells and incubated for an additional 60 min, after which time, the antibody conjugate was removed and the wells were washed with a wash buffer. A substrate, (AttoPhos®, Promega, Madison, Wis.) was added to the wells, and the rate of formation of the fluorescent product was related to the concentration of the marker in the patient samples. In the alternative, markers were measured by commercially available immunoassays on appropriate platforms.

Assays for Thrombus precursor Protein (TpP™) were performed using reagents obtained from American Biogenetic Sciences, Inc., Columbia, Md. Two murine monoclonal antibodies that recognize different epitopes on the soluble fibrin polymer were employed for the assay. The assay was calibrated using TpP™ supplied by American Biogenetic Sciences. Samples were diluted 1:4 prior to assay. The minimal detectable concentration was 0.25 μg/ml and the upper end of the reportable range was 25 μg/ml. Thus, samples between 1 μg/ml and 100 μg/ml would assay in the reportable range.

### Example 3

### Risk Stratification Using TpP™

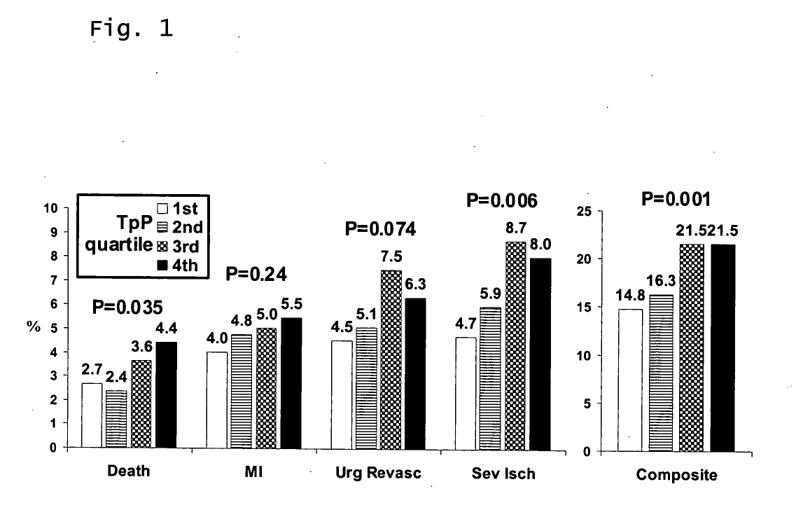

Baseline TpP levels were measured in 2349 patients with ACS enrolled in OPUS-TIMI 16. Death, nonfatal MI, recurrent ischemia requiring urgent revascularization (Urg Revasc), and severe ischemia leading to rehospitalization (Sev Isch) were evaluated thru 10 mos. Median TpP levels were 8.9 μg/ml (IQR 4.8-15.9). Higher baseline levels of TpP were associated with increased age, HTN, DM, prior CAD and prior CHF (P<0.05 for each). TpP levels correlated only weakly with TnI, CRP, & BNP (|r|<0.1 for each). The risk of death, MI, Urg Revasc, Sev Isch, and the composite increased progressively with rising levels of TpP (FIG. 1). After adjusting for baseline differences, TnI, CRP, and BNP, patients with TpP levels above the median remained at significantly increased risk for the composite endpoint (hazard ratio 1.47, p=0.001). In patients with ACS, elevated levels of TpP. at baseline are associated with increased risk of death and ischemic complications. These data support the value of combining a marker of active thrombosis, such as TpP, with established biomarkers of necrosis, inflammation and hemodynamic stress for risk assessment in ACS.

### Example 4

### Exemplary Risk Marker Panels Using TpP™ and MCP-1

The following tables demonstrate the use of methods of the present invention for providing marker panels for risk stratification in ACS. Starting with a number of potential markers, an iterative procedure was applied. In this procedure, individual threshold concentrations for the markers are not used as cutoffs per se. Rather, a “window” of assay values between a minimum and maximum marker concentration (calculated as midpoint ±midpoint x linear range average in the tables below) is determined. Measured marker concentrations above the maximum are assigned a value of 1 and measured marker concentrations below the minimum are assigned a value of 0; measured marker concentrations within the window are linearly interpolated to a value of between 0 and 1. The value is then multiplied by a weighting factor (weight average in the tables below). The absolute values of the weights for all of the individual markers add up to 1. A negative weight for a marker implies that the assay values for the control group are higher than those for the diseased group. A “panel response” is calculated using the midpoint, window, and weighting factors. The panel responses for the entire population of “disease group” and “controls” are subjected to ROC analysis, and a panel response cutoff is selected to yield the desired sensitivity and specificity for the panel. After each set of iterations, the weakest contributors to the equation are eliminated and the iterative process starts again with the reduced number of markers. This process is continued until a minimum number of markers that will still result in acceptable sensitivity and specificity of the panel is obtained.

The following panels represent the combination of markers used to analyze test samples obtained from ACS patients having an adverse event (death, acute myocardial infarction, congestive heart failure, labeled “Disease group”) within 30 days to a “control” group representing ACS patients not having such an event. Negative values indicate that a particular marker decreases in the Disease group as compared to the control group. The column labeled “Ave Resp” indicates the relative contribution of each marker to the final panel response value. An odds ratio was calculated based on these results.

| Panel 1 | | | | Panel 2 | | | |
|---|---|---|---|---|---|---|---|
| | Lin | | | | Lin | | |
| Mid- | Range | Weight | Ave | Mid- | Range | Weight | Ave |
| point | Ave | Ave | Resp | point | Ave | Ave | Resp |
| BNP pg/mL | | | | BNP pg/mL | | | |
| 481.082 | 0.831 | 0.406 | 0.984 | 441.360 | 0.852 | 0.258 | 0.868 |
| cTnI ng/mL | | | | CRP mg/mL | | | |
| 17.120 | 0.695 | 0.314 | 0.855 | 96.637 | 0.526 | 0.318 | 0.823 |
| CRP mg/mL | | | | cTnI ng/mL | | | |
| 136.390 | 0.638 | 0.280 | 0.844 | 15.200 | 0.560 | 0.302 | 0.541 |

| CK-MB ng/mL | | | |
|---|---|---|---|
| 212.145 | 0.416 | 0.122 | 0.110 |

| | | | | |
|---|---|---|---|---|
| Total Controls | 1936 | | 977 | |
| Disease group | 104 | Odds Ratio: | 52 | Odds Ratio: |
| Ave ROC Area | 0.679 | 5.038 | 0.733 | 6.496 |
| SD(%) | 0.006 | | 0.011 | |
| Ave Sens @ 92.5% | 29.0% | | 34.5% | |
| Spec | | | | |
| SD(%) | 1.70 | | 2.40 | |
| Ave Spec @ 92.5% | 23.5% | | 37.9% | |
| Sens | | | | |
| SD % | 2.20 | | 2.00 | |

| Panel 3 | | | | Panel 4 | | | |
|---|---|---|---|---|---|---|---|
| | Lin | | | | Lin | | |
| Mid- | Range | Weight | Ave | Mid- | Range | Weight | Ave |
| point | Ave | Ave | Resp | point | Ave | Ave | Resp |
| MMP-9 ng/mL | | | | BNP pg/mL | | | |
| 132.310 | 0.641 | 0.188 | 0.708 | 333.962 | 0.593 | 0.252 | 0.659 |
| BNP pg/mL | | | | MMP-9 ng/mL | | | |
| 314.837 | 0.586 | 0.218 | 0.605 | 155.742 | 0.646 | 0.185 | 0.637 |
| cTnI ng/mL | | | | cTnI ng/mL | | | |
| 11.868 | 0.489 | 0.178 | 0.385 | 13.639 | 0.571 | 0.256 | 0.454 |
| CRP mg/mL | | | | CRP mg/mL | | | |
| 130.250 | 0.456 | 0.177 | 0.347 | 123.786 | 0.475 | 0.192 | 0.383 |
| Caspase-3 ng/mL | | | | TpP μg/mL | | | |
| −19.555 | −0.337 | −0.239 | 0.262 | 83.850 | 0.493 | 0.115 | 0.291 |

| | | | | |
|---|---|---|---|---|
| Total Controls | 1781 | | 1762 | |
| Disease group | 91 | Odds Ratio: | 92 | Odds Ratio: |
| Ave ROC Area | 0.708 | 5.489 | 0.733 | 5.412 |
| SD(%) | 0.013 | | 0.067 | |
| Ave Sens @ 92.5% | 30.8% | | 30.5% | |
| Spec | | | | |
| SD(%) | 2.00 | | 3.80 | |
| Ave Spec @ 92.5% | 32.1% | | 30.2% | |
| Sens | | | | |
| SD(%) | 2.90 | | 4.60 | |

Panel 6

| | | | |
|---|---|---|---|
| | Lin Range | Weight | |
| Midpoint | Ave | Ave | Ave Resp |

BNP pg/mL

| | | | |
|---|---|---|---|
| 288.355 | 0.592 | 0.195 | 0.607 |

MMP-9 ng/mL

| | | | |
|---|---|---|---|
| 122.311 | 0.657 | 0.157 | 0.567 |

Myoglobin ng/mL

| | | | |
|---|---|---|---|
| 431.069 | 0.532 | 0.222 | 0.500 |

MCP-1 pg/mL

| | | | |
|---|---|---|---|
| 444.400 | 0.659 | 0.134 | 0.325 |

CRP mg/mL

| | | | |
|---|---|---|---|
| 103.747 | 0.516 | 0.115 | 0.290 |

TpP μg/mL

| | | | |
|---|---|---|---|
| 50.137 | 0.499 | 0.108 | 0.209 |

cTnI ng/mL

| | | | |
|---|---|---|---|
| 9.242 | 0.564 | 0.069 | 0.208 |

| | | |
|---|---|---|
| Total Controls | 1710 | |
| Disease group | 90 | Odds Ratio: |
| Ave ROC Area | 0.715 | 7.463 |
| SD(%) | 0.012 | |
| Ave Sens @ 92.5% Spec | 37.7% | |
| SD(%) | 2.60 | |
| Ave Spec @ 92.5% Sens | 33.4% | |
| SD(%) | 3.10 | |

### Example 5

### Diagnosis of Subclinical Atherosclerosis Using MCP-1

MCP-1 has been identified as an independent risk predictor in ACS. See, e.g., de Lemos et al., Circulation 107: 690-95 (2003), which is hereby incorporated by reference in its entirety. The following data demonstrates the use of MCP-1 in the diagnosis of subclinical atherosclerosis. Baseline MCP-1 levels were measured in 3499 patients not exhibiting symptoms of atherosclerosis (based on clinical presentation). A subset of 2733 patients was given electron beam computerized tomography (EBCT) scans. EBCT is an imaging procedure that uses a CT scanner to measure the amount of calcium found in the arteries of the heart. Subclinical coronary artery disease can be detected without the need of surgery or the injection of tracking fluids by measuring coronary artery calcium (“cac”). See, e.g., Khaleeli et al., Am. Heart J. 141: 637-44, 2001.

Distribution of MCP-1 among the 3499 patients from whom it was measured:

| Quartile | N | Median |
|---|---|---|
| 1 (<=123 pg/mL) | 875 | 100.3 [83, 112] |
| 2 (>123.1-167.9 pg/mL) | 877 | 146 [134, 157] |
| 3 (168-226 pg/mL) | 874 | 194 [180, 208] |
| 4 (>226.1 pg/mL) | 873 | 285 [248, 356] |

MCP-1 Levels and Cardiovascular Risk Factors

MCP-1 Quartiles

| Variable | Quartile 1 | Quartile 2 | Quartile 3 | Quartile 4 | P value |
|---|---|---|---|---|---|
| Median | 40 [34, 48] | 43 [36, 51] | 44 [36, 52] | 47 [39, 54] | <0.001 |
| Age(years) | | | | | |
| % patients with | 21.04 | 23.21 | 25.66 | 30.09 | <0.001 |
| hypertension | | | | | |
| % patients with | 18.16 | 24.13 | 26.87 | 30.85 | 0.001 |
| diabetes | | | | | |
| Median Total | 172 [150, 198] | 175 [154, 201] | 180 [156, 204] | 180 [155, 207] | <0.001 |
| Cholesterol | | | | | |
| % patients who | 21.52 | 22.90 | 27.64 | 27.94 | <0.001 |
| are current | | | | | |
| smokers | | | | | |
| % patients with | 22.65 | 24.41 | 25.11 | 27.83 | 0.022 |
| family hx CAD | | | | | |
| Median LDL | 101 [81, 124] | 102 [82, 123] | 106 [84, 128] | 107 [82, 129] | 0.0188 |

These associations are among all 3499 patients.

LDL: Each quartile contains about 875 patients; HTN: 1060 patients had hypertension;

smoking: 1013 patients were current smokers; family hx: 1139 patients had a family h/o cad;

DM: 402 patients had DM.

Associations (not shown) between baseline variables and MCP-1 levels were also performed among the subset of patients that had EBCT scans (n=2733).

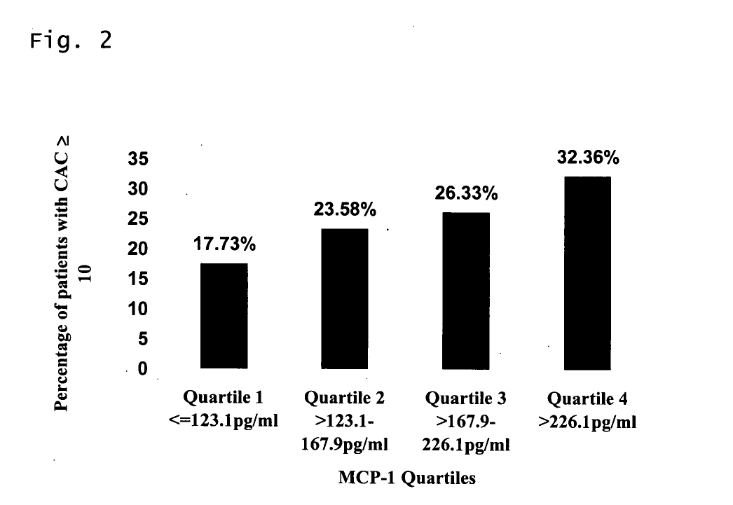

FIG. 2 shows the association of MCP-1 to subclinical atherosclerosis in 2733 patients who had an EBCT scan. Of these, 581 patients had evidence of subclinical atherosclerosis defined as a coronary calcification score ≧10. Additional evidence suggests a significant association between the degree of cac (categorical) and MCP-1 levels (continuous).

Relative Risk for Subclinical Atherosclerosis (CAC≧10) in Multivariate Analysis (Excluding Age)

| Variable | Odds Ratio | P value |
|---|---|---|
| MCP quartile 2 | 1.339 | 0.059 |
| MCP quartile 3 | 1.445 | 0.016 |
| MCP quartile 4 | 1.716 | <0.001 |
| Sex | 2.548 | <0.001 |
| Diabetes | 2.391 | <0.001 |
| Hypertension | 3.425 | <0.001 |
| Tobacco Use | 1.781 | <0.001 |
| Total Chol | 1.521 | 0.009 |

Multivariate Model for Subclinical Atherosclerosis stratified by intermediate or highest age tertile (>=40 years; n=1831)

| Variable | Odds Ratio | P value |
|---|---|---|
| MCP quartile 2 | 1.382 | 0.051 |
| MCP quartile 3 | 1.501 | 0.013 |
| MCP quartile 4 | 1.604 | 0.003 |
| Sex | 2.561 | <0.001 |
| Diabetes | 2.164 | <0.001 |
| Hypertension | 2.424 | <0.001 |
| Tobacco Use | 1.764 | <0.001 |
| Total Chol | 1.322 | 0.099 |
| Family History of CAD | 1.409 | 0.002 |

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of predicting a risk of one or more future clinical outcomes for a subject suffering from a vascular disease, comprising:
determining the presence or amount of thrombus precursor protein in a sample from said subject; and
correlating the presence or amount of thrombus precursor protein to said risk of one or more clinical outcomes for the subject.

2. A method according to claim 1, wherein said vascular disease is selected from the group consisting of acute coronary syndrome, atherosclerosis, ischemic stroke, intracerebral hemorrhage, subarachnoid hemorrhage, transient ischemic attack, systolic dysfunction, diastolic dysfunction, aneurysm, aortic dissections, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis; and peripheral vascular disease.

3. A method according to claim 2, wherein said vascular disease is acute coronary syndrome.

4. A method according to claim 3, wherein said one or more future clinical outcomes are selected from the group consisting of death, nonfatal myocardial infarction, recurrent ischemia requiring rehospitalization, recurrent ischemia requiring urgent revascularization, and congestive heart failure.

5. A method according to claim 4, wherein the correlating step comprises determining the concentration of thrombus precursor protein in said sample, and comparing said concentration to a threshold concentration, wherein a concentration of thrombus precursor protein less than said threshold concentration is indicative of a first risk of said one or more clinical outcomes and a concentration of thrombus precursor protein greater than said threshold concentration is indicative of a second risk of one or more clinical outcomes.

6. A method according to claim 5, wherein said threshold concentration provides a ROC curve area of at least about 0.6.

7. A method according to claim 5, wherein said threshold concentration provides an odds ratio of about 4 or greater or about 0.25 or less.

8. A method according to claim 5, wherein said threshold concentration provides a hazard ratio of about 1.25 or greater or about 0.8 or less.

9. A method according to claim 5, wherein said threshold concentration is a median thrombus precursor protein concentration measured in samples from subjects suffering from acute coronary syndrome.

10. A method according to claim 9, wherein said median thrombus precursor protein concentration is about 8.9 μg/mL.

11. A method according to claim 1, further comprising determining the presence or amount of one or more other subject-derived markers in said sample, and said correlating step comprises correlating the presence or amount of thrombus precursor protein and said one or more other subject-derived markers to said risk of one or more clinical outcomes for the subject.

12. A method according to claim 11, wherein said one or more other subject-derived markers are independently selected from the group consisting of specific markers of myocardial injury, specific markers of neural tissue injury, markers related to blood pressure regulation, markers related to coagulation and hemostasis, markers related to inflammation, and markers related to apoptosis.

13. A method according to claim 12, wherein said one or more other subject-derived markers comprise one or more specific markers of myocardial injury.

14. A method according to claim 12, wherein said one or more other subject-derived markers comprise one or more specific markers of neural tissue injury.

15. A method according to claim 12, wherein said one or more other subject-derived markers comprise one or more markers related to blood pressure regulation.

16. A method according to claim 12, wherein said one or more other subject-derived markers comprise one or more markers related to coagulation and hemostasis.

17. A method according to claim 12, wherein said one or more other subject-derived markers comprise one or more markers related to apoptosis.

18. A method according to claim 12, wherein said one or more other subject-derived markers are selected from the group consisting of annexin V, B-type natriuretic peptide, β-enolase, free cardiac troponin I, complexed cardiac troponin I, free and complexed cardiac troponin I, free cardiac troponin T, complexed cardiac troponin T, free and complexed cardiac troponin T, creatine kinase-MB, glycogen phosphorylase-BB, heart-type fatty acid binding protein, phosphoglyceric acid mutase-MB, S-100ao, adenylate kinase, brain-derived neurotrophic factor, calbindin-D, creatine kinase-BB, glial fibrillary acidic protein, lactate dehydrogenase, myelin basic protein, neural cell adhesion molecule (NCAM), c-tau, neuropeptide Y, neuron-specific enolase, neurotrophin-3, proteolipid protein, S-100β, thrombomodulin, protein kinase C γ, atrial natriuretic peptide (ANP), pro-ANP, B-type natriuretic peptide (BNP), NT-pro BNP, pro-BNP C-type natriuretic peptide, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, urodilatin, acute phase reactants, cell adhesion molecules, C-reactive protein, interleukins, interleukin-1 receptor agonist, monocyte chemoattractant protein-1, caspase-3, lipocalin-type prostaglandin D synthase, mast cell tryptase, eosinophil cationic protein, KL-6, haptoglobin, tumor necrosis factor α, tumor necrosis factor β, Fas ligand, soluble Fas (Apo-1), TRAIL, TWEAK, fibronectin, macrophage migration inhibitory factor (MIF), vascular endothelial growth factor (VEGF), myeloperoxidase, caspase-3, cathepsin D, α-spectrin, plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, and tissue factor, or markers related thereto.

19. A method according to claim 9, wherein said plurality of subject-derived markers comprise BNP or a marker related thereto.

20. A method according to claim 9, wherein said plurality of subject-derived markers comprise free cardiac troponin I, complexed cardiac troponin I, free and complexed cardiac troponin I, free cardiac troponin T, complexed cardiac troponin T, free and complexed cardiac troponin T, or a marker related thereto.

21. A method according to claim 12, wherein said plurality of subject-derived markers comprise C-reactive protein or a marker related thereto.

22. A method according to claim 12, wherein said plurality of subject-derived markers comprise caspase-3 or a marker related thereto.

23. A method according to claim 12, wherein said plurality of subject-derived markers comprise myeloperoxidase or a marker related thereto.

24. A method according to claim 1, wherein the sample is from a human.

25. A method according to claim 1, wherein the sample is selected from the group consisting of blood, serum, and plasma.

26. A method according to claim 1, wherein the assay method is an immunoassay method.

27. A method according to claim 12, wherein the correlating step comprises determining the concentration of thrombus precursor protein and said one or more other subject-derived markers, and individually comparing each concentration to a corresponding threshold level.

28. A method according to claim 12, wherein the correlating step comprises determining the concentration of thrombus precursor protein and said one or more other subject-derived markers, calculating a single index value based on each concentration, and comparing the index value to a threshold level.

29. A method according to claim 1, wherein the method comprises determining a temporal change in thrombus precursor protein concentration, and wherein said temporal change is used in said correlating step.

30. A device for performing the method of claim 12, comprising a plurality of discrete locations, each discrete location configured and arranged to immobilize for detection thrombus precursor protein or one of said one or more other subject-derived markers.

31. The device of claim 30, wherein each of said plurality of discrete spots comprises an antibody that binds thrombus precursor protein or one of said one or more other subject-derived markers.

32. A method of diagnosing atherosclerosis in a subject, comprising:
determining the presence or amount of monocyte chemoattractant protein-1 or a marker related thereto in a sample from said subject; and
correlating the presence or amount of monocyte chemoattractant protein-1 to the presence or absence of atherosclerosis in the subject.

33. A method according to claim 32, wherein the correlating step comprises determining the concentration of monocyte chemoattractant protein-1 or a marker related thereto in said sample, and comparing said concentration to a threshold concentration, wherein a concentration of monocyte chemoattractant protein-1 or a marker related thereto less than said threshold concentration is indicative of a first risk of atherosclerosis and a concentration of monocyte chemoattractant protein-1 or a marker related thereto greater than said threshold concentration is indicative of a second risk of atherosclerosis.

34. A method according to claim 32, wherein said correlating step further comprises determining the presence or amount of one or more risk factors selected from the group consisting of the sex, age, a diagnosis of diabetes, a diagnosis of hypertension, past tobacco use, a cholesterol concentration, and a family history of atherosclerosis, for said subject, wherein the presence or absence of one or more of said risk factors and the presence or amount of monocyte chemoattractant protein-1 or a marker related thereto are correlated to the presence or absence of atherosclerosis in the subject.

35. A method according to claim 33, wherein said threshold concentration provides an odds ratio of about 1.3 or greater or about 0.77 or less.

36. A method according to claim 33, wherein said threshold concentration is selected to provide an odds ratio of about 2 or greater or about 0.5 or less.

37. A method according to claim 33, wherein said median thrombus precursor protein concentration is greater than about 120 pg/mL.

38. A method according to claim 33, wherein said median thrombus precursor protein concentration is greater than about 150 pg/mL.

39. A method according to claim 33, wherein said median thrombus precursor protein concentration is greater than about 200 pg/mL.

40. A method according to claim 32, further comprising determining the presence or amount of one or more other subject-derived markers in said sample, and said correlating step comprises correlating the presence or amount of monocyte chemoattractant protein-1 or a marker related thereto and said one or more other subject-derived markers to the presence or absence of atherosclerosis in the subject.

41. A method according to claim 40, wherein said one or more other subject-derived markers are independently selected from the group consisting of specific markers of myocardial injury, specific markers of neural tissue injury, markers related to blood pressure regulation, markers related to coagulation and hemostasis, markers related to inflammation, and markers related to apoptosis.

42. A method according to claim 32, wherein the sample is from a human.

43. A method according to claim 32, wherein the sample is selected from the group consisting of blood, serum, and plasma.

44. A method according to claim 32, wherein the assay method is an immunoassay method.
